(19) United States
(12) Patent Application Publication
(10) Pub. No.: US 2008/0261905 A1
(43) Pub. Date: Oct. 23, 2008

(54) Modified Nucleosides for Rna Interference

(75) Inventors: Piet Herdewijn, Wezemaal (BE); Arthur Van Aerschot, Heist-Op-Den-Berg (BE); Jing Wang, Sint-Niklaas (BE); Rudy Juliano, Chapel Hill, NC (US)

Correspondence Address: KNOBBE MARTENS OLSON & BEAR LLP, 2040 MAIN STREET, FOURTEENTH FLOOR, IRVINE, CA, 92614, US

(73) Assignees: K.U. LEUVEN RESEARCH AND DEVELOPMENT, Leuven (BE); UNIVERSITY OF NORTH CAROLINA AT CHAPEL HILL, Chapel Hill, NC (US)

(21) Appl. No.: 11/718,793

(22) PCT Filed: Nov. 8, 2005
(86) PCT No.: PCT/BE2005/000159

§ 371 Date: Mar. 13, 2008

(30) Foreign Application Priority Data
Nov. 8, 2004 (GB) 0424600.5
Dec. 10, 2004 (GB) 0427106.0
Dec. 30, 2004 (GB) 0428476.6

(51) Int. Cl. A61K 31/70; C07H 21/00; C07H 21/04; C12N 5/06; C07D 311/00; A61P 43/00

(52) U.S. Cl. 514/44; 536/22.1; 536/23.1; 435/375; 549/397

(57) ABSTRACT

The present invention relates to the use of modified nucleotides and single or double stranded oligonucleotides having at least one of said modified nucleotides for performing RNA interference. The modified nucleotides are selected from 6-membered ring containing nucleotides such as hexitol, altritol, O-substituted or O-alkylated altritol, cyclohexenyl, ribo-cyclohexenyl and O-substituted or O-alkylated ribo-cyclohexenyl nucleotides. The present invention also relates to novel modified nucleosides or nucleotides and to the use of the novel modified nucleosides and nucleotides in single or double stranded oligonucleotides for RNA interference, antisense therapy or other applications.

### FIELD OF THE INVENTION

The present invention relates to modified nucleosides and nucleotides, to nucleotide sequences, oligomers and (oligomer) compositions comprising the same and to their use in gene modulation and in particular RNA interference. The modified nucleotides of the invention are selected from 6-member ring containing nucleotides such as hexitol and cyclohexenyl nucleotides.

### BACKGROUND OF THE INVENTION

Many diseases could be treated and/or cured by inhibiting the expression of specific genes or multiple genes present in an organism, whether they are from endogenous or exogenous origin.

Examples of such diseases are cancer, inherited disorders and infectious diseases. Furthermore, the inhibition of the expression of genes can also be helpful in pharmaceutical target validation and functional genomics.

Methods that disrupt the expression of a certain gene include the antisense, ribozyme and antigene strategy. Recently, a new approach developed in the genetic research area, RNA interference. Post-transcriptional gene silencing, also known as RNA interference (RNAi), is an evolutionary conserved mechanism of gene specific silencing, by which a polynucleotide inhibits the activity of another nucleotide sequence, such as messenger RNA.

This phenomenon has been observed in cells of a diverse group of organisms, including humans, suggesting its promise as a novel therapeutic approach to the genetic control of human disease.

The term RNA-interference (RNAi) has come to generalize all forms of gene silencing involving dsRNA leading to the sequence-specific reduction of endogenous targeted mRNA levels, unlike co-suppression, in which transgenic DNA leads to silencing of both the transgene and the endogenous gene.

RNA interference involves the insertion of small pieces of double-stranded (ds) and even single stranded (ss) RNA into a cell. If the dsRNA corresponds with a gene in the cell, it will promote the destruction of mRNA produced by that gene, thereby preventing its expression.

The technique appears to work on a variety of genes, including those of viruses residing within the cell.

To avoid the non-specific cellular responses to (long) double-stranded RNA in mammalian cells, small interfering RNAs (siRNAs) or short-hairpin RNAs (shRNAs) are designed.

A description of the mechanisms for siRNA activity, as well as some of its applications is described in Provost et al., Ribonuclease Activity and RNA Binding of Recombinant Human Dicer, E. M.B.O.J., 2002 Nov., 1, 21(21): 5864-5874; Tabara et al., The dsRNA Binding Protein RDE-4 Interacts with RDE-1, DCR-1 and a DexH-box Helicase to Direct RNAi in C. elegans, Cell. Jun. 28, 2002, 109(7):861-71; Ketting et al., Dicer Functions in RNA Interference and in Synthesis of Small RNA Involved in Developmental Timing in C. elegans; and Martinez et al., Single-Stranded Antisense siRNAs Guide Target RNA Cleavage in RNAi, Cell Sep. 6, 2002, 110(5):563, all of which are incorporated by reference herein.

The ability to assess gene function via siRNA mediated methods, as well as to develop therapies for over-expressed genes, represents an exciting and valuable tool that will accelerate genome-wide investigations across a broad range of biomedical and biological research.

For in vivo applications, RNAi has been hampered until now by different problems such as the poor stability of RNA (in i.e. blood, serum), the transient nature of the gene suppression.

Consequently, the use of naked siRNA in cell culture, animal studies, and studies aimed at developing therapeutics, has limited potential benefits.

Progress has been made in other applications towards developing modified ribonucleic acids that exhibit improved stability under the above-described conditions, while maintaining some of the nucleic acid's functionality.

Known modifications for these applications include, for example, fluoro, 2′-O-methyl, amine and deoxy modifications at the 21 position of the sugar ring. However, to date there has been only limited focus on the use and optimization of these and other modifications in connection with RNAi.

One limitation on the use of known modifications is that although they increase stability, this benefit comes at a price. For example, some modifications decrease functionality, thereby requiring higher effective doses; others eliminate functionality entirely, and still others are toxic.

Thus, it is clear that there remains a need to develop compositions and methods of using functional stabilized polynucleotides that retain potency or show an increased potency.

Several recent publications have described the structural requirements for the dsRNA trigger required for RNAi activity. Recent reports have indicated that ideal dsRNA sequences are 21 nucleotides in length containing 2 nucleotides 3′-end overhangs (Elbashir et al, EMBO (2001), 20, 6877-6887, Sabine Brantl, Biochimica et Biophysica Acta, 2002, 1575, 15-25.) In this system, substitution of the 4 nucleosides from the 3′-end with 2′-deoxynucleosides has been demonstrated to not affect activity.

On the other hand, substitution with 2′-deoxyoucleosides or 2′-OMe-nucleosides throughout the sequence (sense or antisense) was shown to be deleterious to RNAi activity.

Investigation of the structural requirements for RNA silencing in C. elegans has demonstrated modification of the internucleotide linkage (phosphorothioate) to not interfere with activity (Parrish et al., Molecular Cell, 2000, 6, 1077-1087.) It was also shown by Parrish et al., that chemical modification like 2′-amino or 5-iodouridine are well tolerated in the sense strand but not the antisense strand of the dsRNA suggesting differing roles for the 2 strands in RNAi.

Base modification such as guanine to inosine (where one hydrogen bond is lost) has been demonstrated to decrease RNAi activity independently of the position of the modification (sense or antisense). Some position independent loss of activity has been observed following the introduction of mismatches in the dsRNA trigger.

Some types of modifications, for example introduction of sterically demanding bases such as 5-iodoU, have been shown to be deleterious to RNAi activity when positioned in the antisense strand, whereas modifications positioned in the sense strand were shown to be less detrimental to RNAi activity.

As was the case for the 21 nt dsRNA sequences, RNA-DNA heteroduplexes did not serve as triggers for RNAi. However, dsRNA containing 2′-F-2′-deoxynucleosides appeared to be efficient in triggering RNAi response independent of the position (sense or antisense) of the 2′-F-21 deoxynucleosides.

In one study the reduction of gene expression was studied using electroporated dsRNA and a 25 mer morpholino oligomer in post implantation mouse embryos (Mellitzer et al., Mehanisms of Development, 2002, 118, 57-63). The morpholino oligomer did show activity but was not as effective as the dsRNA.

In a specific study, the inclusion of a 5′-phosphate moiety was shown to enhance activity of siRNA's in vivo in Drosophila embryos (Boutla, et al., Curr. Biol., 2001, 11, 1776-1780). In another study, it was reported that the 5′-phosphate was required for siRNA function in human HeLa cells (Schwarz et al., Molecular Cell, 2002, 10, 537-548).

In yet another recently published paper (Chin et al., Molecular Cell, 2002, 10, 549-561) it was shown that the 5′-hydroxyl group of the siRNA is essential as it is phosphorylated for activity while the 3′-hydroxyl group is not essential and tolerates substitute groups such as biotin. It was further shown that bulge structures in one or both of the sense or antisense strands either abolished or severely lowered the activity relative to the unmodified siRNA duplex.

Also shown was severe lowering of activity when psoralen was used to cross-link an siRNA duplex.

International patent application WO2004/043979 describes the use of sugar modified oligomeric compounds for use in RNA interference.

### SUMMARY OF THE INVENTION

The present invention relates to modified nucleosides and nucleotides (also referred to as nucleoside and nucleotide analogs) with a sugar surrogate moiety and to oligonucleotides comprising said modified nucleosides and nucleotides, especially for using in RNA interference applications.

The terms “nucleosides” and “nucleotides” are used in their general context as known in the prior art (a nucleoside referring to a sugar or sugar surrogate coupled to a heterocyclic ring, mostly a purine or pyrimidine base, while a nucleotide refers to a nucleoside coupled to a phosphate group (or analogs thereof) as present as a monomeric unit in an oligomer or oligonucleotide.

In the context of the invention these nucleosides and nucleotides are also referred to as “6-membered ring nucleosides and nucleotides”, “6-membered ring containing nucleosides and nucleotides” or “6-membered sugar-surrogate containing nucleosides or nucleotides”. In these compounds the 5-membered furanose ring (that is normally present) is replaced by a 6-membered ring. Apart from that replacement other modifications may be possible, such as of the base or internucleotide linkage.

Said 6-membered ring replacing the furanose may be selected from 6-membered sugar rings (substituted or unsubstituted) such as hexoses, but especially may be selected from substituted or unsubstituted ring-oxygen-comprising cyclohexanes (or tetrahydropyran). Preferred ring-oxygen-comprising cyclohexanes are hexitol, altritol, substituted altritols such as (C₃) O-substituted altritols, and more specifically (C₃) O-alkylated altritols. Most preferred are altritol and alkylated altritol (see below).

A preferred ring-oxygen-comprising cyclohexane nucleoside or nucleotide of the invention is one according to the formula I (inclusive salts, esters and isomers thereof),

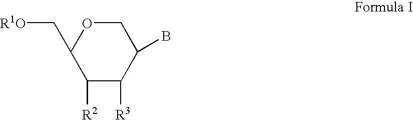

wherein

B is a substituted or unsubstituted heterocyclic ring;

R¹ is independently selected from H, an internucleotide linkage to an adjacent nucleotide or a terminal group;

R² is independently selected from phosphate, any modification known for nucleotides to replace the phosphate group, or from an internucleotide linkage to an adjacent nucleotide or a terminal group;

R³ is independently selected from H, alkyl, alkenyl, alkynyl, azido, F, Cl, I, substituted or unsubstituted amino, OR⁴, SR⁴, aroyl, alkanoyl or any substituent known for modified nucleotides;

R⁴ is selected from hydrogen; alkyl; alkenyl; alkynyl; acyl; wherein said alkyl, alkenyl and alkynyl can contain one or more heteroatoms in or at the end of the hydrocarbon chain, said heteroatom selected from O, S and N.

For preferred compounds according to formula I, R³ is H (hydrogen) or OH (hydroxyl). The respective compounds are hexitol (H) and altitrol (OH) nucleotides respectively.

Other preferred compounds according to formula I are O-substituted or O-alkylated altritols, wherein R³ is OR⁴. Preferably R⁴ is a C₁₋₇ alkyl, most preferably a methyl or ethyl group. R⁴ may also be —CH₂—CH₂—O—CH₃, —CH₂—CH₂—CH₂—NH₂, —CH₂—CH₂—NH₂ and other substituents known in the art.

Preferred isomers are given by formulas Ia-g (see further).

Said 6-membered ring may also be selected from substituted or unsubstituted cyclohexenyls.

A preferred cyclohexenyl nucleotide of the invention is one according to the formula II (inclusive salts, esters and isomers thereof),

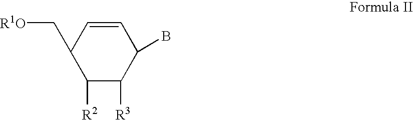

wherein

B is a substituted or unsubstituted heterocyclic ring;

R¹is independently selected from H, an internucleotide linkage to an adjacent nucleotide or a terminal group;

R² is independently selected from phosphate, any modification known for nucleotides to replace the phosphate group, or from an internucleotide linkage to and adjacent nucleotide or a terminal group;

R³ is independently selected from H; OH; OR⁴,

R⁴ is selected from alkyl; alkenyl; alkynyl and acyl; wherein said alkyl, alkenyl and alkynyl can contain one or more heteroatoms in or at the end of the hydrocarbon chain, said heteroatom selected from O, S and N.

Preferred compounds according to formula II are cyclohexenyl (R³ is H), ribo-cyclohexenyl (R³ is OH) and O-substituted ribo-cyclohexenyl (R³ is OR⁴).

Preferred isomers are given by formulas IIa-c (see further).

A preferred cyclohexenyl nucleoside or nucleotide or substituted cyclohexenyl nucleoside or nucleotide of the invention is a C₂-substituted cyclohexenyl nucleoside or nucleotide, more specifically a ribocyclohexenyl nucleoside or nucleotide or (C₂-)substituted ribocyclohexenyl nucleoside or nucleotide, more specifically according to the formula III (inclusive salts, esters and isomers thereof),

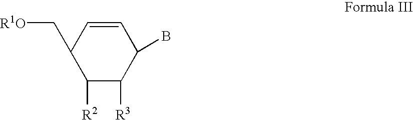

wherein

B is a substituted or unsubstituted heterocyclic ring;

R¹ is independently selected from H; alkyl; alkenyl; alkynyl; acyl; phosphate moieties or a protecting group;

R² is independently selected from OH; O-alkyl; O-alkenyl; O-alkynyl; O-acyl; a O-protecting group; phosphate or any modification known for nucleotides to replace the phosphate group or from an internucleotide linkage to an adjacent nucleotide or a terminal group; wherein said alkyl, alkenyl and alkynyl can contain one or more heteroatoms in or at the end of the hydrocarbon chain, said heteroatom selected from O, S and N;

R³ is independently selected from OH, O-alkyl, O-alkenyl, O-alkynyl, O-acyl or O-protecting group; wherein said alkyl, alkenyl and alkynyl can contain one or more heteroatoms in or at the end of the hydrocarbon chain, said heteroatom selected from O, S and N.

A most preferred O-substituted ribo-cyclohexenyl nucleotide is an O-alkylated ribo-cyclohexenyl, more specifically a C₂—O-alkylated ribo-cyclohexenyl. The alkyl group preferably is a C₁₋₇ alkyl, most preferably a methyl or ethyl group. The alkyl may also contain heteroatoms and may thereby also be —CH₂—CH₂—O—CH₃, —CH₂—CH₂—CH₂—NH₂, —CH₂—CH₂—NH₂ and other substituents known in the art.

Preferred isomers are given by formulas IIIa and b (see further).

Preferred nucleotides of the invention are C₂-substituted cyclohexenyl nucleoside or nucleotide analogs wherein C₂ does not bear 2 hydrogen atoms.

Preferred nucleotides of the invention are amongst others ring-oxygen-comprising cyclohexane nucleotides, such as hexitol nucleotides, altritol nucleotides, O-substituted altritol nucleotides, alkylated altritol nucleotides, and are cyclohexenyl nucleotides, such as ribo-cyclohexenyl nucleotides, O-substituted ribo-cyclohexenyl nucleotides and alkylated ribo-cyclohexenyl nucleotides according to any of the above definitions and formulas.

The present invention particularly relates to novel compounds such as the above ribo-cyclohexenyl nucleotides, O-substituted ribo-cyclohexenyl nucleotides, more specifically O-alkylated ribo-cyclohexenyl nucleotides, especially those according to formulas III and IIIa and b, to the corresponding nucleosides, to nucleotide sequences (of any length), oligomers or (oligomer) compositions comprising these and their different applications such as antisense therapy, modulation of gene expression and in particular RNA interference. The present invention also concerns such nucleoside, nucleotide, or oligomers comprising at least one such nucleotide for use as a medicament and concerns the use of such nucleoside, nucleotides, oligomers and compositions for the preparation of a medicament to treat or prevent cancer, and in general a disease or a disorder associated with a target nucleic acid.

In the case of a nucleoside (for any of the above formulas and definitions) R² is hydroxyl (OH) or OR⁴, provided that R⁴ is not phosphate or analogs thereof. The corresponding nucleosides form another aspect of the invention.

B in any of the above preferably is selected from pyrimidine and purine bases, more specifically from uracyl, adenine, cytosine, thymine and guanine. Adenine is the most preferred base in the case of the ribo-cyclohexenyl nucleotides, O-substituted ribo-cyclohexenyl nucleotides and alkylated ribo-cyclohexenyl nucleotides of the invention.

The invention further relates to nucleotide sequences (also comprising oligonucleotides or polynucleotides), preferably oligomers that comprise at least one 6-membered ring containing nucleotide according to the invention (any of the above).

The nucleotide sequences, preferably oligomers of the invention may be single stranded. In that case they preferably are antisense oligomeric sequences.

The single stranded oligonucleotides, preferably oligomers of the invention may comprise one such 6-membered ring containing nucleotide, or two, three, four or more of such nucleotides. They may for instance contain (comprise) for more than 10%, 20%, 30% or 50% of such 6-membered ring containing nucleotides, compared to other modified nucleotides or normal nucleotides (with a furanose sugar moiety). The complete oligomer (oligomeric strand) may be composed of 6-membered ring containing nucleotides. Most preferably, however, they comprise one (exactly one) 6-membered ring containing nucleotide.

Double stranded oligomers are preferred for use in RNA interference. Such double stranded oligomers are also referred to as duplex oligomers. Preferably they are linear but they may also be circular. The term “double stranded” in the present context includes (oligomeric) hairpin constructs, in particular short-hairpins. The term “double stranded” also includes duplex oligomers (or short-hairpins) with an overhang. In other words, the double stranded oligomers or duplexes according to the invention do not need to be 100% double stranded in the strict sense.

Another aspect of the invention concerns compositions comprising two oligomeric strands (a first and a second oligomer) or two oligomeric regions (a first and a second region), said oligomeric strands/regions being capable of forming e.g. a duplex oligomer or a hairpin construct.

In that context, a preferred composition of the invention is one that comprises a first oligomer and a second oligomer in which at least a portion of the first oligomer is capable of hybridizing with at least a portion of the second oligomer, and at least a portion of the first oligomer is complementary to and capable of hybridizing to a selected target nucleic acid, wherein at least one of said first or said second oligomers includes at least one 6-membered ring containing nucleotide of the invention. Preferably the 6-membered ring containing nucleotide is capable of forming a base pair with a nucleotide of the other oligomer. Preferably, if both the first and the second oligomer comprise such nucleotide, they are in different positions (id est they do not face each other). Recently micro RNA (miRNA) has been discovered. The two hybridizing regions of the miRNA (hairpin) are not 100% complementary, yet these molecules are effective and suited for RNA interference. It is thus not necessary that the first and the second oligomers or first and second regions in a duplex oligomer (as further defined herein) are 100% complementary. The first oligomer has to be complementary to a certain degree with at least a portion of the second oligomer, the percentage of (overall) complementarity of both oligomers or both regions preferably being at least 50%, 60%, preferably at least 70%, 80%, or more preferably at least 90%.

Preferably, the first and second oligomers comprise a complementary pair of siRNA oligomers.

Most preferably, the first and second oligomers comprise an antisense/sense pair of oligomers.

Most preferably the first oligomer in this composition is an antisense oligomer. The second oligomer preferably is a sense oligomer. Preferably the second oligomer has a plurality of ribose nucleoside units.

Preferably, each of the first and second oligomers have 10 to 40 nucleobases, more preferably 18 to 30 nucleobases, yet more preferably 18 to 24 nucleobases, most preferably 21 to 24 nucleobases. It may be preferred to have an overhang, e.g. a 3′ overhang, as previously indicated.

In a duplex oligomer according to the invention, at least one of the oligomers (the first or the second) is modified and contains at least one 6-membered ring containing nucleotide of the invention. If only one oligomer (the first or the second) is modified, it preferably is the antisense strand that is modified and contains (comprises) at least one 6-membered ring containing nucleotide. The term “antisense” herein, in a broad perspective, refers to the strand hybridizing to, complementary to or antisense to part (at least 8 and preferably more nucleotides) of the target nucleotide sequence. However, if only one oligomer (the first or the second) is modified, it can also be the “sense” strand that is modified and contains (comprises) at least one 6-membered ring containing nucleotide.

Very promising results were obtained when both the sense and antisense strand (both the first and the second oligomer) were modified to contain (include or comprise) at least one 6-membered ring containing nucleotide. An aspect of the invention concerns duplex oligomers, wherein the antisense strand contains (comprises) exactly one nucleotide of the invention and the sense strand contains (comprises) at least one (one or more, such as 1, 2, 3, 4, 5, 6, 7, 8, 9, 10, 11, 12, 13, 14, 15, 16, 17, 18, 19, 20 or more) such nucleotide. The invention for instance relates to duplex or double-stranded oligomers with exactly one (only one) 6-membered ring containing nucleotide according to the invention in each strand (in the first oligomer and in the second oligomer).

The sugar-surrogate containing nucleotide of the invention can be present at the middle (in the middle section) of the oligomer (single or duplex), in the 3′- or the 5′-sections, or it can be randomly present or at a specific position within the oligomer.

By the term “present in the 5′-section” is meant in the present context that a nucleoside or nucleotide of the invention is contained within the ten first nucleotides, more preferably within the five first nucleotides starting from the 5′-end of the oligomer, more in particular within the three first nucleotides starting from the 5′-end of the oligomer (single or duplex). By “present in the 3′-section” is meant that a nucleoside or nucleotide of the invention is contained within the ten first nucleotides starting from the 3′-end, more preferably within the five first nucleotides starting from the 3′-end of the oligomer, more in particular within the three first nucleotides starting from the 3′-end of the oligomer (single or duplex). Most preferably the 6-membered ring containing nucleotide of the invention is, however, contained in the middle (middle part or middle section) of the oligomer (single or duplex), more in particular in the oligomer part or section at least 3 nucleotides distant from the 3′- and 5′-end, more in particular at least 5 nucleotides distant from the 3′- and 5′-end, most in particular at least 7 nucleotides distant from the 3′- and 5′-end and finally more in particular at least 9 nucleotides distant from the 3′- and 5′-end. These definitions and positions are given for an oligomer of 21-24 nucleotides and more in particular for an oligomer of 21 nucleotides. Depending on the length of the oligomer, the numbers/positions herein given will change proportionally.

The nucleotide sequences, preferably oligomers (single or doubled stranded) of the invention may comprise (in total or per strand) one such 6-membered ring containing nucleotide, or two, three or more of such nucleotides. They may for instance contain for (comprise) more than 10%, 20%, 30% or 50% of such 6-membered ring containing nucleotides, compared to other modified nucleotides or normal nucleotides. The complete oligomer (one of both strands, or both strands) may be composed of 6-membered ring containing nucleotides.

Excellent results were surprisingly obtained with duplex or ds oligomers wherein at least one strand but more preferably both strands comprise at least one modified nucleotide in the middle section of the oligomer (oligomeric strand).

Excellent results were obtained when both strands (sense and antisense strand, or first and second oligomer) contained exactly one modified nucleotide of the invention in the middle part of the strand (sense and antisense). The oligomers of the invention may further comprise more (further, additional) modified nucleotides, in the middle section, or in any other section of the strand(s). By “modified” is meant here modified nucleotides of the invention or any other type of modified nucleotide known in the art.

Excellent results were also obtained with duplex oligomers wherein the antisense strand comprised exactly one nucleotide according to the invention and the sense strand more than one (or several) of such nucleotides. Best results were again obtained when the nucleotides of the invention were incorporated in the middle part of the strands (sense and antisense, or first and second oligomer).

As mentioned before, the oligomer of the invention may be in the form of a hairpin or a loop structure. The first and second oligomer are then comprised in one single molecule and the composition hereinabove described will then yield a hairpin such as a short-hairpin (shRNA). The first and second oligomer may then be separated by a spacer sequence.

In that same context, yet another aspect of the invention concerns an oligomer having at least a first region and a second region, wherein said first region of said oligomer is complementary to and capable of hybridizing with said second region of said oligomer, at least a portion of said oligomer is complementary to and capable of hybridizing to a selected target nucleic acid, said oligomer further including (comprising) at least one 6-membered ring containing nucleotide of the invention.

Preferably each of said first and said second regions has at least 10 nucleosides.

Preferably said first region in a 5′ to 3′ direction is complementary to said second region in a 3′ to 5′ direction.

Preferably said oligomer forms a hairpin structure. Said first region of said oligomer may be spaced from said second region of said oligomer by a third region (a spacer region or spacer nucleotide sequence), wherein said third region may comprise at least two nucleosides or nucleotides. Alternatively said said first region of said oligomer may be spaced from said second region of said oligomer by a third region, wherein said third region comprises a non-nucleoside or a non-nucleotide.

What has been said about the position and number of modified nucleotides contained in an oligomer or a composition as described hereinabove, applies equally well to a hairpin construct or to compositions capable of providing a hairpin construct.

Another aspect of the invention concerns an oligomer comprising (or including) exactly one 6-membered ring containing nucleotide of the invention. The oligomer may be single-stranded and is then preferably an antisense strand. The oligomer preferably is double-stranded, certainly when intended for use in RNA interference applications. The modified nucleotide of the invention is preferably contained in the middle (or the middle part) of a strand (one of both or both strands of a duplex oligomer). Preferable each strand of the duplex comprises exactly one modified nucleotide of the invention and preferably this modification is present in the middle part or the middle section of the strand (s).

Yet another aspect of the invention relates to an oligomer or oligomer composition (preferred length given above) that comprises at least one 6-membered ring containing oligonucleotide in the middle part or the middle section of the oligomeric strand(s). Preferably this at least one (one or more) modified nucleotide(s) in the middle is contained in the antisense strand and possibly, in addition thereto, in the sense strand. Most preferably the oligomer is a duplex oligomer, possibly provided by a composition of the invention. The modification herein described may be present in one of the strands, yet preferably is present in both strands. In a preferred embodiment, the antisense strand comprises one (1, exactly one, only 1) such modification and the sense strand at least one such modification. In another preferred embodiment both strands of the duplex comprise exactly one such modification in the middle section of the strand. The 6-membered ring containing nucleotide may be any of the ones described above, but preferably it is a cyclohexenyl nucleotide or a ring-oxygen-comprising cyclohexane nucleotide, a (C₂-) substituted cyclohexenyl nucleotide, and more in particular a ribo-cyclohexenyl nucleotide, a (C₂—)O-substituted ribo-cyclohexenyl nucleotide, a (C₂—)O-alkyl ribo-cyclohexenyl nucleotide, an altritol nucleotide, a (C₃-)substituted altritol nucleotide, a (C₃—)O-substituted altritol nucleotide, or a (C₃—)O-alkyl altritol nucleotide according to any of the above definitions or formulas.

Another aspect of the invention relates to duplex oligomers or to compositions providing these, which comprise at least one modified nucleotide of the invention per strand. Excellent results were obtained when each strand of a duplex oligomer contained exactly one such nucleotide and this preferably in the middle part or the middle section of the strands. Excellent results were further obtained when the antisense strand contained one such nucleotide, and the sense strand contained several (one or more) of such nucleotides. Once more, the nucleotides of the invention are preferentially incorporated in the middle part of the strands. Once more the 6-membered ring containing nucleotide may be any of the ones described above, but preferably it is a cyclohexenyl nucleotide or a ring-oxygen-comprising cyclohexane nucleotide, a (C₂-)substituted cyclohexenyl nucleotide, and more in particular a ribo-cyclohexenyl nucleotide, a (C₂—)O-substituted ribo-cyclohexenyl nucleotide, a (C₂—)O-alkyl ribo-cyclohexenyl nucleotide, an altritol nucleotide, a (C₃—) substituted altritol nucleotide, a (C₃—)O-substituted altritol nucleotide, or a (C₃—)O-alkyl altritol nucleotide according to any of the above definitions or formulas.

Still another aspect of the invention concerns a composition comprising a first oligomer and a second oligomer, wherein: at least a portion of said first oligomer is capable of hybridizing with at least a portion of said second oligomer, at least a portion of said first oligomer is complementary to and capable of hybridizing with a selected target nucleic acid, and wherein said first oligomer and/or said second oligomer include at least one 6-membered ring containing nucleotide selected from the group consisting of ribo-cyclohexenyl nucleotides, (C₂—)O-substituted ribo-cyclohexenyl nucleotides, altritol nucleotides, (C₃—) O-substituted altritol nucleotides or any mixture thereof. The invention further relates to the duplex oligomers formed by such composition. Indications on the strands and their type, length etc have been given earlier. Preferred positions of the modifications have also been given before.

Yet another aspect of the invention concerns a pharmaceutical composition comprising a nucleoside, a nucleotide, an oligomer or a composition according to the invention (any of the above), and a pharmaceutically acceptable carrier.

The oligomers or compositions of the invention are particularly suited for modulation of gene expression, antisense therapy, and in particular for RNA interference.

Another aspect of the invention relates to the use of 6-membered ring containing nucleotides of the invention for the construction of oligomers to be used in RNA interference. Incorporation of nucleotides of the invention in a duplex RNA molecule improved stability while at least maintaining functionality. Functionality mostly even improved. Yet another aspect of the invention therefore relates to a method for improving the stability and/or functionality (for RNA interference) of an oligomer by incorporating at least one nucleotide or nucleoside of the invention.

The oligomers or compositions of the invention are highly suited for RNA interference. Surprisingly, the oligomers of the invention comprising at least one 6-membered ring nucleotide of the invention behaved much better than unmodified oligomers in terms of for instance activity and stability, like nuclease stability.

Another aspect of the invention concerns a method of modulating the expression of a target nucleic acid in a cell, said method comprising the step of contacting said cell with an oligomer or composition according to the invention. Preferably expression of the gene is hereby reduced or gene inhibition is hereby increased. Preferably gene inhibition is increased by at least 5%, 10%, more preferably at least 25%, 30% and most preferably at least 50%, or even at least 75% compared to a control (e.g. compared to treatment with a standard siRNA that does not comprise a nucleotide of the invention). This method of modulation may be an in vitro method. For many oligomers of the invention the effect almost doubled (compared to standard siRNA).

Yet another aspect of the invention concerns a method of treating or preventing a disease or disorder associated with a target nucleic acid, said method comprising the step of administering to e.g. a plant or an animal (preferably a mammal such as a human) having or predisposed to said disease or disorder a therapeutically effective amount of a nucleoside, a nucleotide, an oligomer or a composition according to the invention.

The oligomers and compositions of the invention are particularly suited to treat cancer.

The oligomers and compositions of the invention are further particularly suited to down-regulate the MDR1 gene that is involved in cancer cell drug resistance, and to inhibit or reduce in particular the expression of cell surface P-glycoprotein expression. They are for instance suited to inhibit or decrease the expression of P-glycoprotein efflux pumps.

The invention further concerns to the use of a nucleoside, a nucleotide and in particular an oligomer or composition of the invention for the preparation of a medicament to treat or prevent a disease associated with a target nucleic acid. This disease may be cancer. The nucleotide sequence in question may be e.g. the MDR1 gene involved in cancer cell drug resistance but it may equally well be a viral sequence. Especially oligomers comprising at least one of SEQ ID NOs: 8-30 proved very suitable for these purposes.

The nucleosides, nucleotides, oligomeric compounds or oligomers, and compositions of the invention can additionally be used for diagnostics, therapeutics, prophylaxis and as research reagents and kits. Such uses allow for those skilled in the art to elucidate the function of particular genes or to distinguish between functions of various members of a biological pathway.

The invention will in sequel be described in further detail with reference to the figures, tables and examples, which in no way are intended to be limiting.

### BRIEF DESCRIPTION OF THE DRAWINGS

FIG. 1: NIH 3T3-MDR cells were treated with Lipofectamine 2000 and 50 nM duplex siRNA, namely unmodified siRNA (control siRNA) or modified siRNA comprising cyclohexenyl modified nucleosides in only one oligonucleotide of the duplex. Cell surface P-glycoprotein expression in viable cells was evaluated by immunostaining and flow cytometry. The percentage reductions in P-glycoprotein expression were calculated on the basis of the fraction of the cell population shifted to greater than one standard deviation below the mean of the untreated controls. The number of the modified oligonucleotide used in the experiments is shown in the figure. Sense strand: thin line; antisense strand: thicker line.

FIG. 2: Various concentrations (X-axis in nM) of two duplex siRNAs with cyclohexenyl modified nucleosides in only one oligonucleotide of the duplex were compared to completely unmodified duplex siRNA. Cell surface P-glycoprotein expression in NIH 3T3-MDR cells was measured by flow cytometry as described in the examples.

FIG. 3: MDR cells were treated with 50 nM modified siRNA duplexes comprising cyclohexenyl modified nucleosides in both oligonucleotides ( [symbol] ) or 50 nM unmodified ( [symbol] ) duplexes and cell surface P-glycoprotein expression in the viable cells were evaluated by flow cytometry. The mean and standard deviation are derived from 3 experiments. Sense strand: thin line; antisense strand: thicker line.

FIG. 4: Various concentrations (X-axis in nM) of modified duplex oligonucleotides comprising cyclohexenyl modified nucleosides in both strands were compared to unmodified siRNA. Cell surface P-glycoprotein expression in NIH 3T3-MDR cells was measured by flow cytometry.

FIG. 5: Specificity of MDR1 siRNA duplexes comprising cyclohexenyl modified nucleosides in only one strand or in both strands, measured by real-time PCR analysis. MDR-3T3 cells treated with unmodified siRNA or 50 nM modified oligonucleotides comprising cyclohexenyl modified nucleosides were quantified by real-time PCR. Values were normalized with those of GAPDH and expressed as fold change over untreated cells.

FIG. 6: NIH 3T3-MDR cells were transfected with either siRNA unmodified oligonucleotides, modified siRNA duplexes comprising cyclohexenyl modified nucleosides in only one oligomer (2179) or modified siRNA duplexes comprising cyclohexenyl modified nucleosides in both strands (2179/2186) for 4 hours and then grown for 72 hours in 2% FBS DMEM−H. The cells were then exposed for 24 hours to various concentrations of Adriamycin (doxorubicin). After a further 48 hours in drug free 2% FBS/DMEM−H medium, cell numbers were determined by particle counter and results expressed as percent growth of the untreated control (MDR 3T3).

FIG. 7: measurement of Rhodamine uptake. NIH 3T3-MDR cells were treated with 50 nM either modified siRNA duplexes comprising cyclohexenyl modified nucleosides in only one oligomer (single numbers like 2179, 2181, etc) or modified siRNA duplexes comprising cyclohexenyl modified nucleosides in both strands (double numbers like 2179/2183, etc.) complexed with lipofectamine 2000 as described. As a control untreated NIH 3T3-MDR cells (MDR-3T3) and unmodified oligonucleotides (siRNA) were used. Values of Rhodamine 123 uptake were measured, with the 100% level taken as that for untreated NIH 3T3-MDR cells. Mean and standard errors of 3 determinations.

FIG. 8: Nuclease stability—Lane 1-3 Load standard (90% degradation, 50% degradation, full load). Lane 4-6 control Sense/Antisense siRNA; Lane 7-9 2179/Antisense siRNA; Lane 10-12 Sense/2183 siRNAi; Lane 13-15 2179/2183 siRNAi; Pancreatic Rnase incubations were 15′, 30′, 45′; 10% serum incubations were 12, 24, 72 h.

FIG. 9: MDR cells were treated with 50 nM modified siRNA duplexes comprising hexitol modified nucleosides (HNA), altritol modified nucleosides (ANA), alkylated altritol modified nucleosides (3′-OMe) or 50 nM unmodified duplexes and cell surface P-glycoprotein expression in the viable cells were evaluated by flow cytometry. The mean and standard deviation are derived from 3 experiments.

FIG. 10: Structure of natural “deoxy”-(A) “arabino”-(B) and “ribo” (C) nucleosides and their cyclohexenyl congeners (D-F). The preferred conformation of the “sugar” moiety is indicated. The preferred conformations of the furanose nucleosides (A-C) in solid state is described in reference 11. The preferred conformation of the cyclohexenyl nucleosides is derived from NMR coupling constants as given in table 1.

FIG. 11: The deamination reaction was followed with chiral HPLC using Chiralpak AD column (250×4.6 mm): racemic (±)-rCe-A 18 (a) and the progress of the deamination process (b) and (c).

FIG. 12: Important intraresidue NOE contacts in the cyclohexenyl nucleosides

### DESCRIPTION

The inventors have discovered that especially 6-membered ring nucleotide containing oligomers have a potent activity for RNA interference. Especially hexitol, hexitol derived, cyclohexenyl and cyclohexenyl derived nucleotides proved highly suited for incorporation in oligomers to be used in RNA interference applications. The incorporation of such nucleotides in an oligomer improved its stability without negative or detrimental effect on functionality and with an increased RNA interference activity for most of them.

The present invention provides for the use of 6-membered ring containing oligomeric compounds for gene modulation, specifically through RNA interference. The present invention furthermore provides for a novel modified nucleoside or nucleotide and the use of said novel modified nucleosides and nucleotides in single or double stranded oligonucleotides for RNA interference, antisense therapy, antigene therapy and other purposes such as in diagnostic applications.

A first aspect of the present invention relates to the use of 6-membered ring containing nucleotides or nucleosides for the construction of oligomers to be used in RNA interference. These oligomers for RNA interference may be among others, siRNA, miRNA or shRNA molecules. Another aspect of the present invention relates therefore to the use of oligomers comprising at least one 6-membered ring containing nucleotide for RNA interference. Another aspect of the invention relates to compositions comprising oligomers, wherein at least one nucleoside is a 6-membered ring containing nucleoside. Yet another aspect of the present invention relates to a method of performing RNA interference, said method comprising exposing a double stranded oligomer (polynucleotide or oligonucleotide) to a target nucleic acid, wherein said double stranded oligomer (polynucleotide or oligonucleotide) is comprised of a sense strand and an antisense strand, and wherein at least one of said sense strand and said antisense strand comprises at least one 6-membered ring containing nucleotide.

One aspect of the present invention thus relates to the use of oligomers comprising at least one 6-membered ring containing nucleotide for RNA interference. In a particular embodiment said oligomer comprises one such 6-membered ring containing nucleotide, or two or more or contains for more than 10%, 20%, 30% or 50% of such 6-membered ring containing nucleotides, compared to other modified nucleotides or normal nucleotides. In another embodiment, the complete oligomer or at least one strand thereof is composed of 6-membered ring containing nucleotides.

In a particular embodiment, the present invention relates to the use of oligonucleotides comprising at least one 6-membered ring containing nucleotide for obtaining an increased inhibition of a target gene or oligonucleotide through RNA interference, compared to natural or standard RNA oligonucleotides. In a particular embodiment, such an increase of inhibition is at least a 25% increase, yet more in particular at least a 50% increase, yet more in particular a 75% increase of inhibition of a target gene or oligonucleotide through RNA interference compared to natural RNA oligonucleotides. This increase in inhibition can be measured by the methods described herein.

Yet another particular embodiment of the present invention relates to use of oligomers comprising at least one 6-membered ring containing nucleotides to manufacture a medicament for the prevention or treatment of an animal, preferably a mammal such as a human from a certain disorder or disease through RNA interference. In a particular embodiment, said disorder or disease is cancer.

In a specific embodiment, the 6-membered ring containing nucleoside/nucleotide of the invention has an aglycone 6-membered ring sugar-surrogate, which in a more particular embodiment is a 1,5-anhydrohexitol ring. These nucleosides/nucleotides or the invention are referred to as ring-oxygen-comprising cyclohexane or tetrahydropyran and hexitol nucleosides/nucleotides respectively. In a particular embodiment, the 6-membered ring containing nucleoside or nucleotide is a substituted or unsubstituted 1,5-anhydrohexitol nucleoside analogue, wherein the 1,5-anhydrohexitol is coupled via its 2-position to a heterocyclic ring, more specifically a purine or pyrimidine base. In a particular embodiment, the 1,5-anhydrohexitol is substituted at the 3-position, more specifically with R³ as defined hereinbelow.

Below some typical examples of nucleotides according to the invention are given. The structure of the corresponding nucleoside can be derived therefrom by a person skilled in the art.

In certain embodiments, the 6-membered nucleotides are of the formula I (and salts, esters and isomers thereof),

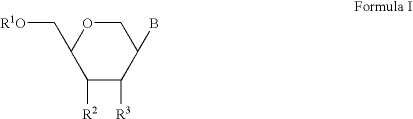

wherein

B is a substituted or unsubstituted heterocyclic ring;

R¹ is independently selected from H, an internucleotide linkage to an adjacent nucleotide or a terminal group;

R² is independently selected from phosphate, from any modification known for nucleotides to replace the phosphate group, from an internucleotide linkage to and adjacent nucleotide or a terminal group;

R³ is independently selected from H, alkyl, alkenyl, alkynyl, azido, F, Cl, I, substituted or unsubstituted amino, OR⁴SR⁴, aroyl, alkanoyl or any substituent known for modified nucleotides;

R⁴ is selected from hydrogen; alkyl; alkenyl; alkynyl; acyl; wherein said alkyl, alkenyl and alkynyl can contain one or more heteroatoms in or at the end of the hydrocarbon chain, said heteroatom selected from O, S and N.

In a particular embodiment, R³ is hydrogen. In another particular embodiment, R³ is OH. They are referred to as hexitol (R³ is H) or altritol (R³ is OH) nucleotides (terms used as in EP0646125 or WO02/18406).

In a yet preferred embodiment, the 6-membered ring containing nucleotide is according to formula I hereinabove, wherein R³ is selected from OR⁴. Such compound is also referred to as an O-substituted altritol nucleotide. In yet another particular embodiment, R⁴ is selected from alkyl, more particularly from C₁₋₇ alkyl, most particularly it is methyl. Thereby, in a preferred embodiment of this invention, the 6-membered sugar surrogate containing nucleotide is an alkylated altritol nucleotide (R³ is O-alkyl). Alkylated and O-substituted altritol nucleotides are examples of altritol derived nucleotides.

In another embodiment, the 6-membered ring containing nucleotide is selected from the formulas Ia, Ib and Ic hereunder

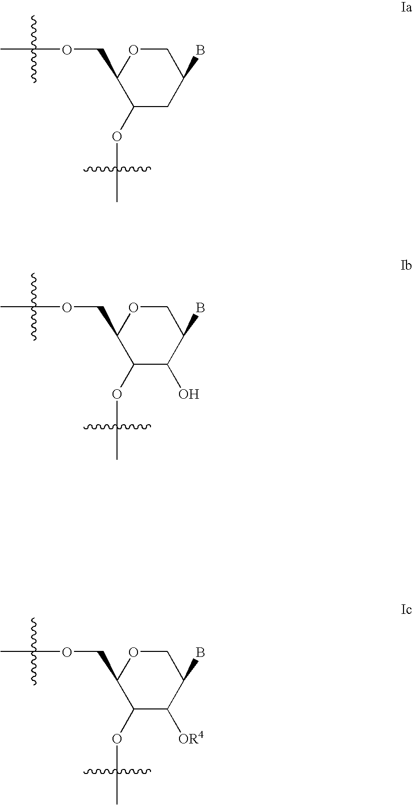

wherein B and R⁴ are as hereinabove described (see formula I and the above paragraphs for the most preferred R⁴ substituents).

In a particular embodiment, the hexitol of the 1,5-anhydrohexitol nucleotide analogues of the invention has the D-configuration and/or the B, R² and R³ of the 1,5-anhydrohexitol nucleoside analogues have the (S)-configuration.

In another embodiment, the 6-membered ring containing nucleotide is selected from the formulas Id, Ie and If hereunder

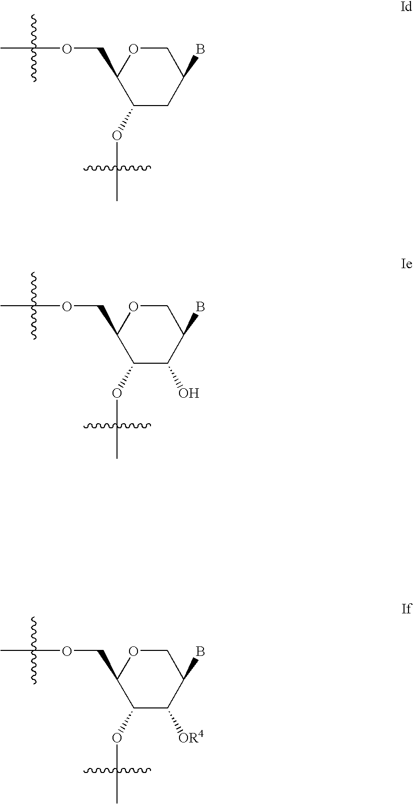

An aspect of the present invention thus relates to the use of oligomers comprising at least one 6-membered ring containing nucleotides for RNA interference, wherein said 6-membered ring containing nucleotide comprises the following unsubstituted or substituted formula Ig:

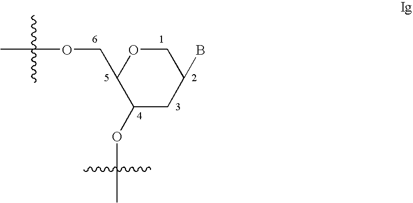

In another specific embodiment, the 6-membered ring containing nucleotide of the invention is selected from 6-membered rings which are substituted or unsubstituted cyclohexenyl nucleotides (also referred to as cyclohexenyl and cyclohexenyl derived nucleotides).

In another embodiment of the present invention, the cyclohexenyl nucleotides are of the formula II (salts, esters and isomers thereof),

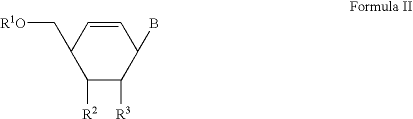

wherein

B is a substituted or unsubstituted heterocyclic ring;

R¹ is independently selected from H, an internucleotide linkage to an adjacent nucleotide or a terminal group;

R² is independently selected from phosphate, any modification known for nucleotides to replace the phosphate group, or from an internucleotide linkage to and adjacent nucleotide or a terminal group;

R³ is independently selected from H; OH; O-alkyl; O-alkenyl; O-alkynyl; or O-acyl; wherein said alkyl, alkenyl and alkynyl can contain one or more heteroatoms in or at the end of the hydrocarbon chain, said heteroatom selected from O, S and N.

A particular embodiment hereof relates thus to the use of oligomers comprising at least one 6-membered ring containing nucleotide for RNA interference, wherein said 6-membered ring containing nucleotide comprises the following formula IIa

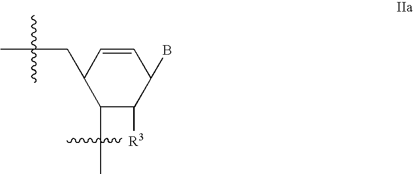

In a particular embodiment, R³ is hydrogen and thus the 6-membered ring containing nucleotide is a cyclohexenyl nucleotide.

In another particular embodiment, R³ is OH and thus the 6-membered sugar surrogate containing nucleotide is a ribo-cyclohexenyl nucleotide.

In yet another particular embodiment, R³ is O-alkyl, yet more specifically is O—C₁₋₇ alkyl, most particularly is O-methyl. These compounds are also referred to as O-substituted and O-alkylated ribo-cyclohexenyl nucleotides. Ribo-cyclohexenyl nucleotides and the different O-substituted forms are examples of cyclohexenyl derived nucleotides.

In a particular embodiment, B is selected from pyrimidine and purine bases, yet more specifically from uracyl, adenine, cytosine or guanine. In another particular embodiment, the cyclohexenyl nucleoside or nucleotide has the D-(like)-configuration.

In another embodiment, the 6-membered ring containing nucleoside/nucleotide is selected from the formulas IIb and IIc hereunder

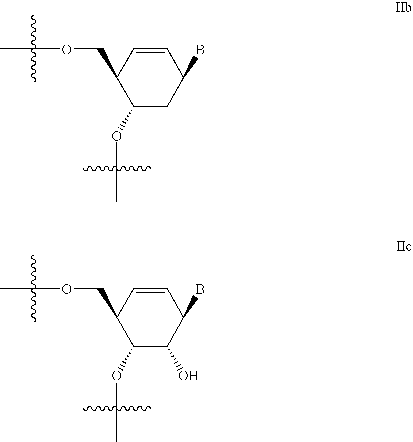

wherein B is as described herein (see formula II and see the previous paragraph for most preferred R⁴ substituents).

For all embodiments of the invention, B can be selected in a specific embodiment from substituted or unsubstituted purine or pyrimidine heterocyclic rings or bases or yet in a more specific embodiment from adenine, guanine, cytosine, uracil, thymine or hypoxanthine.

Another aspect of the present invention relates to compositions comprising a first oligomer and a second oligomer in which at least a portion of the first oligomer is capable of hybridizing with at least a portion of the second oligomer, and at least a portion of the first oligomer is complementary to and capable of hybridizing to a selected target nucleic acid, wherein at least one of said first or said second oligomers includes at least one 6-membered ring containing nucleotide, more in particular capable of forming a base pair with a nucleotide of the other oligomer.

In a particular embodiment, the first and second oligomers comprise a complementary pair of siRNA oligomers.

In certain embodiments, the first and second oligomers comprise an antisense/sense pair of oligomers. Each of the first and second oligomers have 10 to 40 nucleobases in some preferred embodiments. In other embodiments, each of the first and second oligomers have 18 to 30 or 18 to 24 nucleobases. In yet other embodiments, the first and second oligomers have 21 to 24 nucleobases or nucleosides or nucleotides.

Certain aspects of the invention concern compositions in which the first oligomer is an antisense oligomer. In these aspects, the second oligomer is a sense oligomer. In certain preferred embodiments, the second oligomer has a plurality of ribose nucleoside units.

In a particular embodiment, the modified oligomers can be the sense or the antisense strand or both strands are modified. The 6-membered ring containing nucleotides can thus be present in the sense or in the antisense strand of a RNAi duplex. In a preferred embodiment, the antisense oligomer of a specific siRNA duplex is modified in a way that it contains at least one 6-membered ring containing nucleotide, more preferably exactly one such nucleotide.

Another particular embodiment of the present invention relates to the use of double stranded oligonucleotides (sense and antisense strand) wherein at least one oligonucleotide strand comprises at least one 6-membered ring containing nucleotides for RNA-interference. In a particular embodiment, only the sense strand comprises at least one 6-membered ring containing nucleotide, more preferably exactly one or two such nucleotide(s). In another particular embodiment, only the antisense strand comprises at least one 6-membered ring containing nucleotide, more preferably exactly one such nucleotide. In a preferred embodiment, both strands, the sense and the antisense strand of the double stranded oligonucleotides comprise at least one 6-membered ring containing nucleotide. In a particular embodiment, both strands of a duplex for RNA interference comprise exactly 1 modified nucleoside/nucleotide of the invention, so one modified nucleoside/nucleotide per strand. Alternatively, the antisense strand comprises exactly one modified compound according to the invention, whereas the sense strand comprises several (one or more, e.g. 1, 2, 3, 4, . . . ) such compounds.

In some embodiments, at least one oligomeric strand includes a 6-membered ring containing nucleotide. The 6-membered sugar surrogate can be in the first oligomer. In other compounds, the 6-membered sugar surrogate can be in the second oligomer. In yet other embodiments, the sugar surrogate can appear in both the first and second oligomers. The 6-membered ring containing nucleotides can be present at the middle or in the middle section of the oligomer, can be present at the 3′ or 5′ ends, can be present in the 3′- or 5′-section or can be randomly present in the oligomers or at a specific position within the oligomer. In a particular embodiment, the modified nucleotides are present at the 5′-end or in the 5′-section of the oligonucleotide, more in particular within the ten first nucleotides from the 5′-end, yet more specifically within the five first nucleotides of the oligonucleotide from the 5′-end. In another particular embodiment, the modified nucleosides are present at the 3′-end or in the 3′-section of the oligonucleotide, more in particular within the ten first nucleotides from the 3′-end, yet more specifically within the five first nucleotides of the oligonucleotide from the 3′-end. In yet another more particular embodiment, the modified nucleotides are present in the middle or the middle section of the oligonucleotide, more in particular at a position at least 3 nucleotides distant from the 3′- and 5′-end, yet more in particular at least 5 nucleotides distant from the 3′- and 5′-end, yet more in particular at least 7 nucleotides distant from the 3′- and 5′-end and finally more in particular at least 9 nucleotides distant from the 3′- and 5′-end. This counts for an oligomer of about 21 nucleobases. If of a different length, the above indications change proportionally.

Hereinbelow the “middle section” can be defined by way of an example for an oligomer of 100 nucleobases (100 nucleobases=100% of the nucleobases). By the “5′section” is then meant the first 50 (50% of the) nucleobases, preferably the first 25 (25% of the) nucleobases, most preferably the first 10 (10% of the) nucleobases (counting started from the 5′ end). By the “3′section” is then meant the first 50 (50% of the) nucleobases, preferably the first 25 (25% of the) nucleobases, most preferably the first 10 (10% of the) nucleobases (counting started from the 3′ end). The “middle section” or the “middle part” of an oligomer is then defined as the section or the part of the oligomer from the 11^{th} to the 90^{th} nucleobase, preferably from the 21^{th} to the 80^{th} nucleobase, the 26^{th} to the 75^{th} nucleobase, more preferably from the 31^{st} to the 70^{th} nucleobase, from the 36^{th} to the 65^{th} nucleobase, from the 41^{st} to the 60^{th} nucleobase, most preferably from the 46^{th} to 55^{th} nucleobase, from the 48^{th} to the 53^{th} nucleobase (counting started from the 5′ end). Id est 10%, preferably 15%, 20%, 25%, more preferably 30%, 35%, 40%, 45%, 47% of the nucleobases lies respectively to the left and the right of nucleobases contained in the middle section of the oligomer.

In general, the modified nucleotide of the invention can be contained within the 3′-section, within the 5′-section, within the middle section or be present at any given position. Hereinbelow some preferred embodiments are given. In a particular embodiment, the modified nucleosides or nucleotides of the invention are not present in the first 10% nucleotides of the oligonucleotide, starting from the 5′-end and/or from the 3′-end. In another particular embodiment, the modified nucleosides or nucleotides are present in the first 20%, yet more particularly within the first 25% nucleotides in the oligonucleotide, starting from the 5′-end and/or from the 3′-end, even more specifically between the first 10 to 25% nucleotides of an oligonucleotide. In another particular embodiment, the modified nucleosides or nucleotides are present in the middle or the middle section of an oligonucleotide, so between the first 25 to 75% nucleotides of an oligonucleotide, yet more in particular between the first 30-70%, yet more in particular between first 35-65%, yet more in particular between first 40 to 60% nucleotides of an oligonucleotide, always starting from the 5′-end or from the 3′-end. Yet more specifically, the modified nucleosides/nucleotides are not present in the first 25% nucleotides of an oligomer starting from the 5′-end and/or from the 3′-end. In another particular embodiment, the modified nucleosides/nucleotides are present in the sense oligonucleotide in the first 75%, yet more particularly the first 50%, yet more particularly, the first 30% nucleotides, starting from the 3′-end.

In another particular embodiment, the modified nucleosides or nucleotides of the invention are present in the first 10% nucleotides in the oligonucleotide, starting from the 5′-end and/or from the 3′-end. In a particular embodiment, the first or the two first nucleotides of an oligomer at the 5′- and/or 3′-end are modified nucleotides as described herein.

In another particular embodiment, the modified nucleoside/nucleotide is on position 9, 10, 11, 12 or 13 of a 21-mer oligonucleotide. In another particular embodiment, the modified nucleoside/nucleotide is present on position 4 or 5 of the sense oligonucleotide starting from the 3′-end.

Another aspect of the present invention relates to a novel modified nucleoside or nucleotide analog, said novel modified nucleoside or nucleotide analog being according to formula III, isomers or (pharmaceutically acceptable) salts or esters thereof,

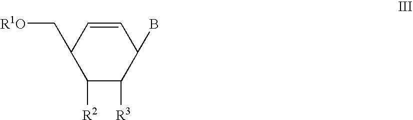

wherein

B is a substituted or unsubstituted heterocyclic ring;

R¹ is independently selected from H; alkyl; alkenyl; alkynyl; acyl; phosphate moieties or a protecting group;

R² is independently selected from OH; O-alkyl; O-alkenyl; O-alkynyl; O-acyl; a O-protecting group phosphate; any modification known for nucleotides to replace the phosphate group; or from an internucleotide linkage to and adjacent nucleotide or a terminal group; wherein said alkyl, alkenyl and alkynyl can contain one or more (1, 2, 3, 4 or more) heteroatoms in or at the end of the hydrocarbon chain, said heteroatom selected from O, S and N;

R³ is independently selected from OH; O-alkyl; O-alkenyl; O-alkynyl; O-acyl or O-protecting group; wherein said alkyl, alkenyl and alkynyl can contain one or more (1, 2, 3, 4 or more) heteroatoms in or at the end of the hydrocarbon chain, said heteroatom selected from O, S and N.

Thus the present invention relates to C₂-substituted cyclohexenyl nucleoside or nucleotide analogs wherein C₂ does not bear two hydrogen atoms.

A particular embodiment of this aspect of the invention relates to a novel modified nucleoside or nucleotide analog, said novel modified nucleotide or nucleoside being according to formula III a or b

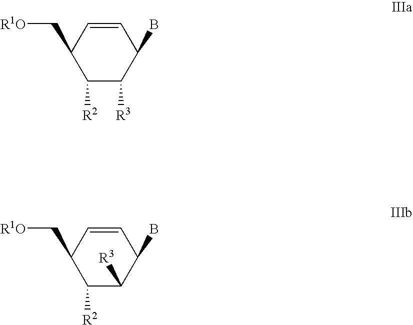

### Wherein

B is a substituted or unsubstituted heterocyclic ring;

R¹ is independently selected from H; alkyl; alkenyl; alkynyl; acyl; phosphate moieties or a protecting group;

R² is independently selected from OH (in case of a nucleoside), phosphate or any modification known for nucleotides to replace the phosphate group, or from an internucleotide linkage to and adjacent nucleotide or a terminal group;

R³ is independently selected from OH; O-alkyl; O-alkenyl; O-alkynyl; O-acyl or O-protecting group; wherein said alkyl, alkenyl and alkynyl can contain one or more (1, 2, 3, 4 or more) heteroatoms in or at the end of the hydrocarbon chain, said heteroatom selected from O, S and N.

This novel modified nucleoside or nucleotide analog comprises a 2′-substituted cyclohexenyl sugar surrogate moiety. In a particular embodiment, the novel nucleosides or nucleotides comprise a 2′-OH cyclohexenyl sugar surrogate moiety, so wherein R³ in formula III is OH. In another particular embodiment, R¹ is hydrogen and R² is OH. In yet another particular embodiment, B is selected from purine and pyrimidine bases, yet more in particular from adenine, guanine, thymine, cytosine, hypoxanthine and uracil.

In a particular embodiment, the novel C₂-substituted cyclohexenyl nucleoside or nucleotide analogs are of the D-like-configuration. In another particular embodiment, the C₂ bearing substituent is in the (S)-configuration or yet more in particular in the (R)-configuration. In a particular embodiment, the novel compounds of the invention are chirally pure.

A particular embodiment of the present invention relates to the compound selected from the group of (±)-(1R,2S,3R,6R)-3-(aden-9-yl)-6-(hydroxymethyl)-4-cyclohexene-1,2-diol, (±)-(1R,2S,3R,6R)-3-(guan-9-yl)-6-(hydroxymethyl)-4-cyclohexene-1,2-diol, (±)-(1R,2S,3R,6R)-3-(thymin-1-yl)-6-(hydroxymethyl)-4-cyclohexene-1,2-diol, (±)-(1R,2S,3R, 6R)-3-(cytosine-1-yl)-6-(hydroxymethyl)-4-cyclohexene-1,2-diol and (±)-(1R,2S,3R,6R)-3-(uracil-1-yl)-6-(hydroxymethyl)-4-cyclohexene-1,2-diol or from their chirally pure D-(like)-configuration nucleoside analogs.

Another aspect of the present invention relates to certain novel intermediates that are made and used during the course of manufacturing one or more of the C₂-substituted cyclohexenyl nucleosides of the formula III, IIIa or IIIb. Such novel intermediates may be represented by the following general formulae IV, V (also Va, Vb and Vc), VI to VII and VIIIa:

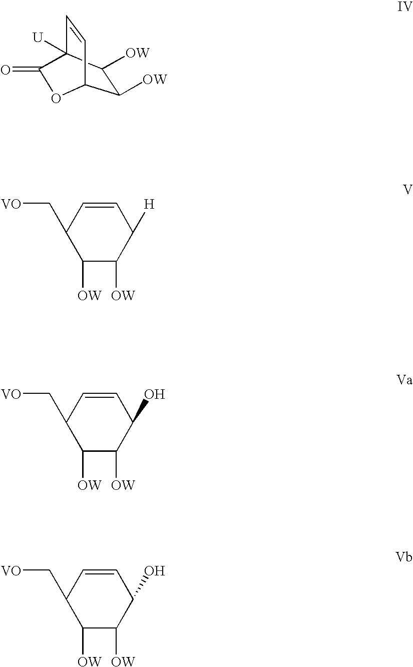

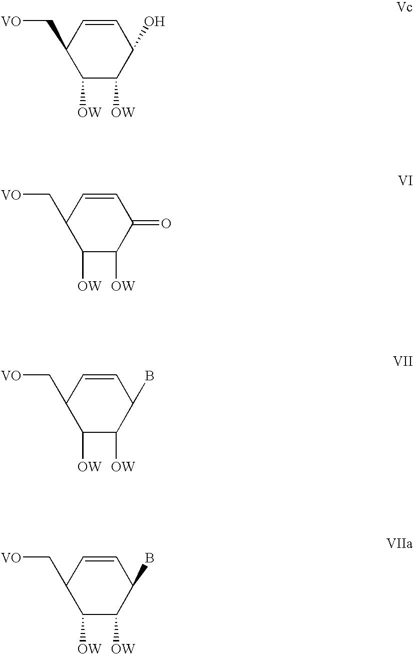

wherein

U is selected from hydrogen and halogen such as Br;

W represents a protecting group, which can be an acetal or ketal protecting the neighbouring diol such as an isopropylidene or benzylidene;

V is selected from hydrogen or a protecting group such as tert-butyldimethylsilyl;

B is selected from a substituted or unsubstituted heterocyclic ring.

In a preferred embodiment, the invention relates to the intermediates Vb, Vc and VI.

The present invention relates in a particular embodiment to novel compounds and intermediates selected from the group consisting of:
1-Bromo-4,4-dimethyl-3,5,8-trioxa-tricyclo[5.2.2.0] undec-10-en-9-one;
4,4-Dimethyl-3,5,8-trioxa-tricyclo[5.2.2.0]undec-10-en-9-one
(±)-(3aS,4R,7R,7aR)-7-(Hydroxymethyl)-2,2-dimethyl-3a,4,7,7a-tetrahydro-1,3-benzodioxo-4-ol
(±)-(3aS,4R,7R,7aR)-7-({Tert-butyl(dimethyl)silyl]oxy}methyl)-2,2-dimethyl-3a,4,7,7a-tetrahydro-1,3-benzodioxo-4-ol
(±)-(3aS,4R,7R,7aR)-7-({Tert-butyl(dimethyl)silyl]oxy}methyl)-2,2-dimethyl-7,7a-dihydro-1,3-benzodioxo-4(3aH)-one
(±)-(3aS,4S,7R,7aR)-7-({[Tert-butyl(dimethyl)silyl]oxy}methyl)-2,2-dimethyl-3a,4,7,7a-tetrahydro-1,3-benzodioxo-4-ol
(±)-9-[(3aS,4R,7R,7aR)-7-({[Tert-butyl(dimethyl)silyl]oxy}methyl)-2,2-dimethyl-3a,4,7,7a-tetrahydro-1,3-benzodioxol-4-yl]-9H-purin-6-amine.

Another aspect of the present invention relates to a process for providing a compound, isomers and a pharmaceutically acceptable salts and esters thereof according to formula III, IIIa or IIIb, said process comprising use of any of the compounds IV to VII, including IV, V (also Va, Vb and Vc), VI to VII and VIIIa.

Another aspect of the present invention relates to oligonucleotides or oligomers comprising the novel modified nucleotides of the present invention. Said oligonucleotides comprise at least one nucleotide, said nucleotide comprising a 6′-substituted cyclohexenyl sugar-surrogate moiety.

Another aspect of the present invention relates to a pharmaceutical composition comprising an oligomer which comprises at least one compound according to formula III, IIIa or 111b herein. It may further comprise other oligomers or compounds according to the invention.

### DETAILED DESCRIPTION

### Definitions

The terms “nucleosides”, “nucleotides”, “oligomers”, “hybridization”, “complementary”, “target sequences”, “targeting”, “sites that may be targeted”, “preferred target segements”, “overhangs”, “oligomers”, “oligomeric compounds”, “oligonucleotides”, “modulation of gene expression” and the like are in general lines (and unless otherwise specified or further detailed) as used in WO 2004/043979.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “6-membered ring containing nucleoside” or “6-membered ring containing nucleotide” refers to modified nucleosides, resp. nucleotides in which at least the furanose ring of the nucleosides/nucleotides are modified in a 6-membered ring such as ring-oxygen-comprising cyclohexan (or cyclohexyl or tetrahydropyran), cyclohexyl or cyclohexenyl and other 6-membered ring systems, such as in hexitols, altritols and cyclohexenyls. The terms refer to 6-membered sugar-surrogate ring comprising nucleosides or nucleotides.

The term “hexose” refers to six-membered cyclic monosaccharides.

The terms “hexitol” and “altritol” refer to their designation in literature as in EP0646125 for hexitol. With altritol is referred to the building blocks of ANA (altritol nucleic acids) comprising a D-altritol backbone and in a particular embodiment with the heterocyclic ring, more specifically the nucleobase, in a 2-(S)-position of the carbohydrate residue.

The term “heterocyclic ring” refers to any ring system comprising heteroatoms such as N, O and S, and wherein the ring system can be substituted or unsubstituted. The term heterocyclic ring therefore comprises the purine and pyrimidine bases, thus the purines and pyrimidines, such as adenine, cytosine, uracyl, thymine or guanine.

The term “pyrimidine and purine bases” or “heterocycle selected from the group consisting of pyrimidine and purine bases” include but are not limited to adenine, thymine, cytosine, uracyl, guanine and (2,6-)diaminopurine and analogues thereof. A purine or pyrimidine base is a purine or pyrimidine base found in naturally occurring nucleosides as mentioned above. An analogue thereof is a base which mimics such naturally occurring bases in that their structures (the kinds of atoms and their arrangement) are similar to the naturally occurring bases but may either possess additional or lack certain of the functional properties of the naturally occurring bases. Such analogues include those derived by replacement of a CH moiety by a nitrogen atom, e.g. 5-azapyrimidines such as 5-azacytosine) or vice versa (e.g., 7-deazapurines, such as 7-deazaadenine or 7-deazaguanine) or both (e.g., 7-deaza, 8-azapurines). By derivatives of such bases or analogues are meant those bases wherein ring substituents are either incorporated, removed, or modified by conventional substituents known in the art, e.g., halogen, hydroxyl, amino, C₁₋₆ alkyl. Such purine or pyrimidine bases, analogues and derivatives are well known to those skilled in the art. In a particular embodiment, the term “pyrimidine and purine bases” or “heterocycle selected from the group consisting of pyrimidine and purine bases” refers to adenine, thymine, cytosine, uracyl and guanine. In another particular embodiment, the purine and pyrimidine bases are substituted with specific groups for a specific function. Specific embodiments of bases B suitable for inclusion into the compounds of the present invention include, but are not limited to, hypoxanthine, guanine, adenine, cytosine, inosine, thymine, uracil, xanthine, 8-aza derivatives of 2-aminopurine, 2,6-diaminopurine, 2-amino-6-chloropurine, hypoxanthine, inosine and xanthine; 7-deeza-8-aza derivatives of adenine, guanine, 2-aminopurine, 2,6-diaminopurine, 2-amino-6-chloropurine, hypoxanthine, inosine and xanthine; 1 deaza derivatives of 2-aminopurine, 2,6-diaminopurine, 2-amino-6-chloropurine, hypoxanthine, inosine and xanthine; 7-deaza derivatives of 2-aminopurine, 2,6diaminopurine, 2-amino-6-chloropurine, hypoxanthine, inosine and xanthine; 3 deaza derivatives of 2-aminopurine, 2,6-diaminopurine, 2-amino-6-chloropurine, hypoxanthine, inosine and xanthine; 6-azacytosine; 5-fluorocytosine; 5 chlorocytosine; 5-iodocytosine; 5-bromocytosine; 5-methylcytosine; 5 bromovinyluracil; 5-fluorouracil; 5-chlorouracil; 5-iodouracil; 5-bromouracil; 5 trifluoromethyluracil; 5-methoxymethyluracil; 5-ethynyluracil and 5-propynyluracil.

The term “alkyl” as used herein refers to C1-C18 normal, secondary, or tertiary hydrocarbon chains. Examples are methyl, ethyl, 1-propyl, 2-propyl, 1-butyl, 2-methyl-1-propyl(1-Bu), 2-butyl(s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl.

The term “alkenyl” as used herein is C2-C18 normal, secondary or tertiary hydrocarbon with at least one site (usually 1 to 3, preferably 1) of unsaturation, i.e. a carbon-carbon, sp2 double bond. Examples include, but are not limited to: ethylene or vinyl (—CH═CH₂) and allyl (—CH₂CH═CH₂). The double bond may be in the cis or trans configuration.

The terms “alkynyl” as used herein refer respectively C2-C18 normal, secondary or tertiary hydrocarbon with at least one site (usually 1 to 3, preferably 1) of unsaturation, i.e. a carbon-carbon, sp triple bond. Examples include, but are not limited to: acetylenic (—C≡CH) and propargyl (—CH₂C≡CH).

The terms “C₁₋₁₈ alkylene” as used herein each refer to a saturated, branched or straight chain hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The terms “alkenylene” as used herein refer to an unsaturated branched chain, straight chain hydrocarbon radical of 2-18 carbon atoms, and having two monovalent radical centers derived by the removal of two hydrogen atoms from the same or two different carbon atoms of a parent alkene, i.e. double carbon-carbon bond moiety. Typical alkenylene radicals include, but are not limited to: 1,2-ethylene (—CH═CH—).

The terms “alkynylene” as used herein refer respectively to an unsaturated, branched or straight chain of 2-18 carbon atoms, having two monovalent radical centers derived by the removal of two hydrogen atoms from the same or two different carbon atoms of a parent alkyne, i.e. triple carbon-carbon bond moiety. Typical alkynylene radicals include, but are not limited to: acetylene (—C≡C—), propargyl (—CH₂C≡C—), and 4-pentynyl (—CH₂CH₂CH₂C≡CH—).

By way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or 9-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated, unsaturated or aromatic ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. Carbocycle thus includes some aryl groups.

The term “acyl” as used herein refers to substituted C(O), such as C(O) (alkyl, alkenyl, alkynyl, phenyl or aryl, such as for example an alkanoyl group (alkylcarbonyl, alkyl coupled to a carbonyl), an aroyl group (arylcarbonyl, aryl attached to a carbonyl), a arylalkanyl or a alkylaryl group, wherein the C(O) is coupled to another molecule or atom and wherein said alkyl, alkenyl and alkynyl can contain a heteroatom in or at the end of the hydrocarbon chain, said heteroatom selected from O, S and N. As an example the term “acyloxyalkyl” refers to an acyl, coupled via an oxygen to alkyl, wherein the alkyl will be further coupled to another atom.

As used herein and unless otherwise stated, the terms “C₁₋₁₈ alkoxy”, “thio C₁₋₇ alkyl”, refer to substituents wherein a C₁₋₁₈ alkyl radical (each of them such as defined herein), are attached to an oxygen atom or a sulfur atom through a single bond, such as but not limited to methoxy, ethoxy, propoxy, butoxy, thioethyl, thiomethyl, and the like.

As used herein and unless otherwise stated, the term halogen means any atom selected from the group consisting of fluorine (F), chlorine (Cl), bromine (Br) and iodine (I).

For the nucleoside/nucleotide analogs herein referred to as hexitol or altritol analogs or derivatives thereof, as for example represented by the formula I, the numbering of the ring structure will be as following:

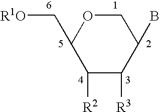

Alternatively, for the nucleoside/nucleotide analogs herein referred to as cyclohexenyl nucleosides/nucleotides, as for example represented by the formula II, the numbering of the ring structure will be as following:

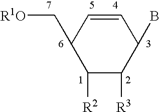

The term “oligomer” as used herein refers to a sequence of nucleotides coupled to each other and it comprises the term “oligonucleotide”. Within the oligonucleotide or oligomer, the phosphate groups are commonly referred to as forming the intersugar backbone or internucleotide linkage of the oligonucleotide or oligomer. The normal linkage or backbone of RNA and DNA is a 3′ to 5′ phosphodiester linkage. Oligonucleotides or oligomers may comprise nucleotide sequences sufficient in identity and number to effect specific hybridization with a particular nucleic acid. In the context of the invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of another oligonucleotide, then the oligonucleotide and the other oligonucleotide are considered to be complementary to each other at that position. The oligonucleotide and the DNA or RNA are complementary to each other when a sufficient number of corresponding positions in each molecule are occupied by nucleotides which can hydrogen bond with each other. “Specifically hybridizes” and “complementary” are thus terms which are used to indicate a sufficient degree of complementarity or precise pairing such that stable and specific binding occurs between the oligonucleotides that hybridize.

The term “isomer” as used herein means all possible isomeric forms, including tautomeric and stereochemical forms, which the compounds according to the formulas of the application like (I), (II), (III) may possess, but not including position isomers. Typically, the structures shown herein exemplify only one tautomeric or resonance form of the compounds, but the corresponding alternative configurations are contemplated as well, including enantiomers and diastereoisomers. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Bliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compounds of the invention. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCeI™ CA, OA, OB5, OC5, OD, OF, OG, OJ and OK, and Chiralpak™ AD, AS, OP(±) and OT(±). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The term “salt” as used herein refers to salt forms of the compounds which appear during the synthesis procedure. The term “pharmaceutically acceptable salts” as used herein means the therapeutically active non-toxic salt forms which the compounds according to the formulas of the application like (I), (II), (III) are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butandioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds according to the formulas of the application like (I), (II), (III) as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄⁺(wherein X is C1-C4 alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄⁺ (wherein X typically is independently selected from H or a C1-C4 alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

The terminology “an internucleotide linkage to an adjacent nucleotide” as used herein means that the compound is coupled via that specific position to an adjacent molecule, said adjacent molecule being a nucleoside or nucleotide in an oligomer. Normally, in a non-modified DNA or RNA oligomer, said internucleotide linkage is a phosphate group. However, in the prior art, many modifications of the phosphate groups are known as internucleotide linkage, such as phosphorothioate. The term “internucleotide linkage” refers also to said modified linkages as known to a person skilled in the art.

The term “terminal group” as used herein means any terminal group known to a person skilled in the art for a terminal group at the 5′- or 3′-end of an oligomer, such as an acyl group, such as acetyl.

The terminology “alkyl; alkenyl; alkynyl; wherein said alkyl, alkenyl and alkynyl can contain one or more heteroatoms in or at the end of the hydrocarbon chain, said heteroatom selected from O, S and N” refers to hydrocarbon chains comprising one or more heteroatoms in the hydrocarbon chain, such as in —CH₂—O—CH₃, —CH₂—O—CH₂—CH₃, —CH₂—CH₂—O—CH₃, —CH₂—CH₂—CH₂—NH₂, —CH₂—CH₂—NH₂, —CH₂—CH₂—O—N(CH₃)₂. More specifically said term can refer to —O—-[(CH₂)ₓ₁—O]ₓ₂-E or —[(CH₂)ₓ₁—O]ₓ₂-E wherein x1 is selected from 2 to 6 (2, 3, 4, 5 or 6); X2 from 0 to 6 (0, 1, 2, 3, 4, 5, 6); and E is C₁-C₆ alkyl or N(Q₁) (Q₂); wherein each Q₁ and Q₂ are independently selected from hydrogen, C₁-C₆ alkyl, substituted alkyl, a nitrogen protecting group (wherein Q₁ and Q₂ can be taken together) and E is hydrogen provided x2 is different from zero.

The term “protection group” as used herein refers to a chemical group used in a synthesis strategy to temporary protect a certain functionality like a hydroxy group or a nitrogen atom and are well known in the art. Examples include but are not limited to TBDMS, benzoyl, benzyl, benzylidene, acyl, acetyl, monomethoxytrityl or isopropylidene.

As used herein and unless otherwise stated, the snake-like symbol through a bond as in the formula Ia means that the bond is part of a bond to another atom in a bigger molecule, more in particular refers to a monomeric unit in an oligomer.

Any substituent designation that is found in more than one site in a compound of this invention shall be independently selected.

### DETAILED DESCRIPTION

Chemically modified siRNA's were tested for their silencing capacity. In particular hexitol nucleotides (HNA), cyclohexenyl nucleotides (CeNA) and altritol nucleotides (ANA), eventually with a 3′-O-methyl substituent (3′-Q-methyl), were tested by incorporating them in siRNAs. They were compared with standard unmodified siRNA of the same sequence. The target used was the MDR1 gene that is involved in cancer cell drug resistance. The gene product is the P-glycoprotein that is expressed on the cell surface. Pgp expression was monitored using a fluor-tagged anti-Pgp monoclonal antibody and flow cytometry. A ‘left-shift’ of the flow profile indicates a reduced Pgp expression. The siRNAs are transfected into the cells by standard means using Lipofectamine 2000. Initial studies showed that 50 nM siRNA gave a strong but partial left shift. Thus all the modified siRNAs were compared at this dose. As seen in the flow profiles above, several of the modified siRNAs gave a stronger ‘left shift’ than did unmodified siRNA, and especially the altritol containing siRNAs yielded a much stronger silencing than the unmodified siRNAs and even than the other modified siRNAs. In the summary Table 1 this is expressed as % reduction in Pgp expression versus an untreated control; we also show the difference between the modified and control siRNA (Minus siRNA control) as well as an indication of cell toxicity (obtained by cell counting). The increased effects shown by the modified siRNAs are significant, especially since mostly only a single position was modified.

RNA interference involves mostly the insertion of small pieces of double-stranded (ds) RNA into a cell. If the dsRNA corresponds with a (target) gene in the cell, it will promote the destruction of (target) mRNA produced by that gene, thereby preventing its expression. It has to be clear to a person skilled in the art that for RNA interference preferably duplexes are used, meaning that two (oligomeric) strands hybridize to each other. These two strands in a duplex can be two separate oligomers or two separate oligomeric strands. Most preferably double stranded linear RNA molecules are used. The duplex can, however, also be formed by one single oligomer of which two parts hybridize with each other such as in hairpin oligomers or hairpin (oligomeric) constructs (shRNA). Recently, it has been found that microRNA (miRNA) can be applied for RNA interference. mRNA is an approximately 22-nucleotide RNA strand which are found in the genomes of animals and plants. They are cleaved from a precursor miRNA and can form a duplex hairpin although not with 100% complementary regions (with multiple mismatches). They can be used for RNA interference and can thereby also contain the modified nucleosides as described herein. Therefore, it is not necessary that the first and the second oligomers or regions in a duplex oligomer are 100% complementary. miRNA forms hairpins wherein 100% complementary regions or strands are present, is well suited for RNA interference. The first oligomer is complementary for a certain percentage with at least a portion of the second oligomer, said percentage being between 50%, 60%, 70%, 80%, or 90%. The oligomers can have multiple mismatches such as 2, 3, 4, 5, 6, 7, 8 and more.

### Biological Activity of CeNA Modified RNA Duplexes.

As biological model we chose to down-regulate the MDR1 gene which is involved in cancer cell drug resistance. The gene product is the P-glycoprotein that is expressed on the cell surface. Pgp expression is monitored using a fluor-tagged anti-Pgp monoclonal antibody and flow cytometry. The siRNA mimics are transfected into the cells by standard means using Lipofectamine 2000. We have introduced cyclohexenyl-A and a cyclohexenyl-G nucleotides in the sense and in the antisense strand at different. A single cyclohexenyl nucleoside was incorporated at the 5′-end of the sense strand (entry 2176) and at nucleotide position −6 (GS 2177), −10 (GS 2178), −17 (GS 2179) and −18 (GS 2181) of the sense strand (counting from the 5′-end). In the antisense strand, a modification was introduced at the −2 (GS 2185), −4 (GS 2186), −8 (GS 2183) position (counting for the 3′-end and not including the dTdT overhang). In three examples, two cyclohexenyl nucleosides were incorporated in the same sequence (entry 2180 and 2182 of the sense oligo and entry 2184 of the antisense oligo). The synthesized modified RNA's are given in example 2.

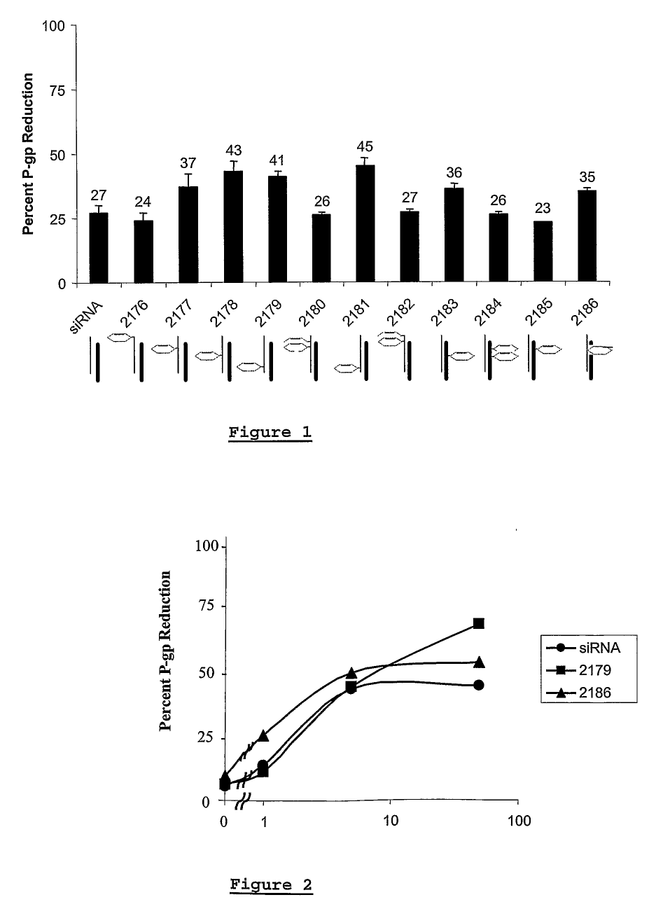

In initial experiments, duplexes of CeNA modified oligonucleotides were formed with the complementary unmodified RNA and used as siRNAs at 50 nM. The percentage P-glycoprotein reduction was measured as described in herein and compared with unmodified siRNA duplexes. In previous experiments we have shown that mismatched or ‘irrelevant’ siRNAs do not affect P-glycoprotein expression levels Xu, D., et al. Mol. Pharmacol. 2004, 66, 268-275. The current results are given in FIG. 1. All CeNA containing duplexes show similar or increased biological activity when compared to the unmodified duplexes. This is most striking for duplexes containing the cyclohexenyl nucleoside in the middle section of the sense sequence (GS 2177, 2178, 2179) and the antisense sequence (GS 2183, 2186). SiRNA with modifications in the end regions (GS 2176, 2180, 2182, 2185) of both sequences does not have a beneficial effect on the biological activity. 3′-End modification with cyclohexenyl nucleoside seems to be better accommodated in the sense sequence (GS 2181) than in the antisense sequence (GS 2185). Two oligonucleotide duplexes were selected for more intensive dose-response studies, one with a modified nucleoside in the sense sequence (GS 2179) and another with the modified nucleoside in the antisense sequence (GS 2186) (FIG. 2). The antisense modified siRNA shows increased biological activity over the whole dose-range, while the sense modified siRNA became more effective at higher concentrations.

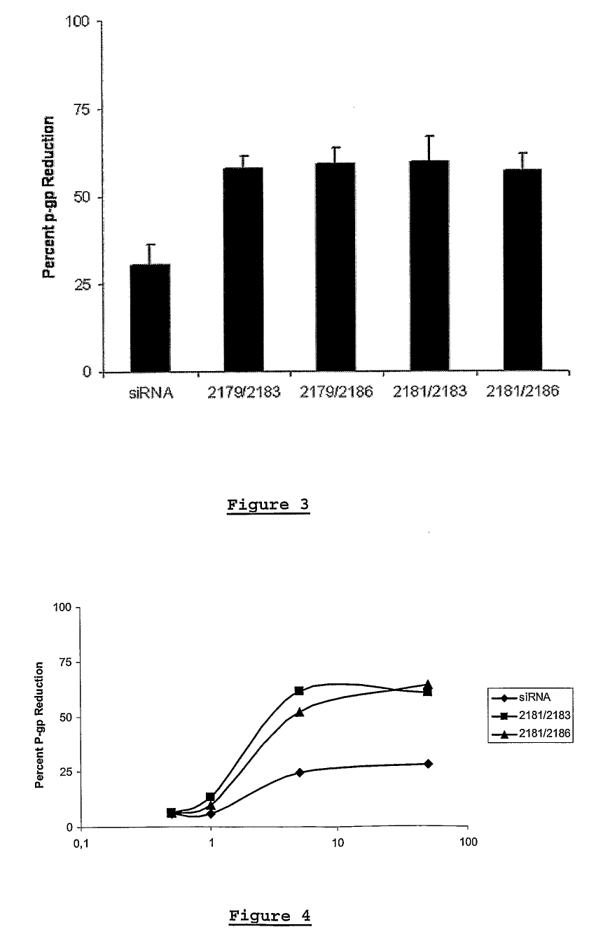

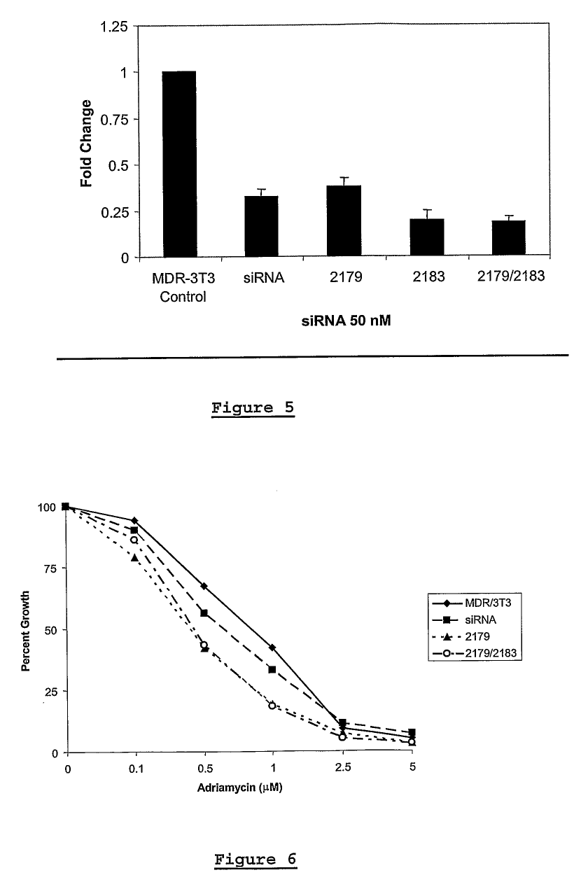

Next, we examined whether the effect of sense and antisense modification of siRNA with cyclohexenyl nucleic acids is additive with modifications in both strands (FIG. 3). Equivalents of the G-modified RNA's of GS 2179 and 2183 and the A-modified RNA's of GS 2181 and 2186 were used for duplex formation. Also modified A/G mixed siRNA were obtained by duplex formation between oligonucleotides of GS 2179 and GS 2186, and of GS 2181 and 2183. In all cases, the biological activity increased by about 100% (at 50 nM siRNA concentration) when compared with natural double stranded RNA. Dose-response curves of two of these duplexes (FIG. 4) shows that this effect is uniform over the whole dose range. The duplex 2179/2183 was also used to demonstrate that the biological activity parallels a decrease in mRNA concentration. Therefore total RNA was extracted from cells, transcribed into cDNA and quantified by real-time PCR. As can be observed in FIG. 5, the modifications in both strands indeed produce the best effect.

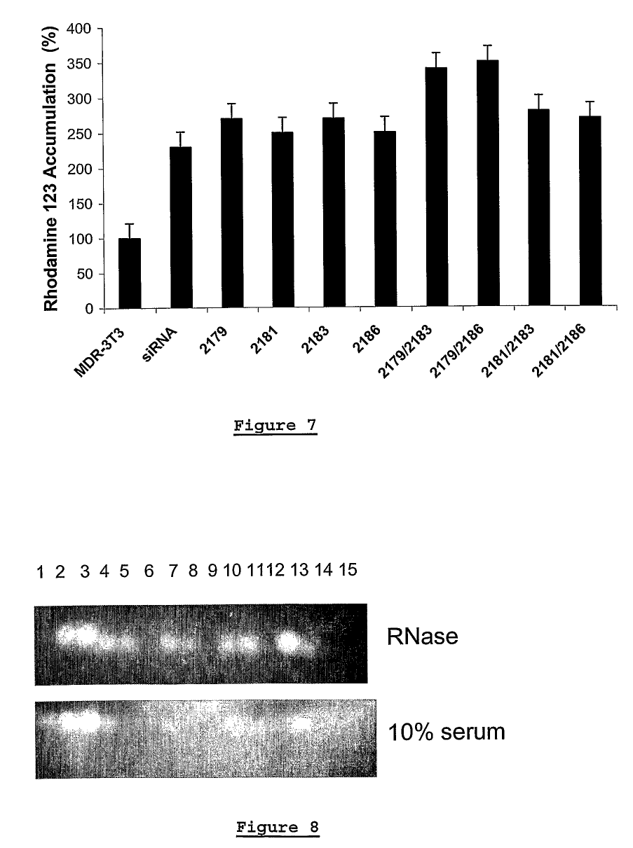

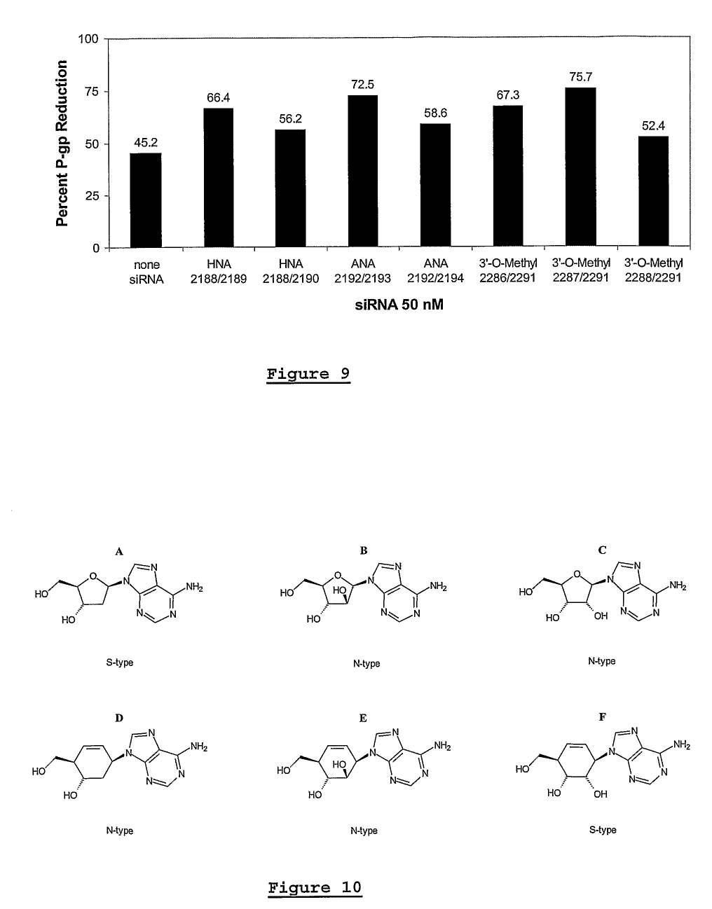

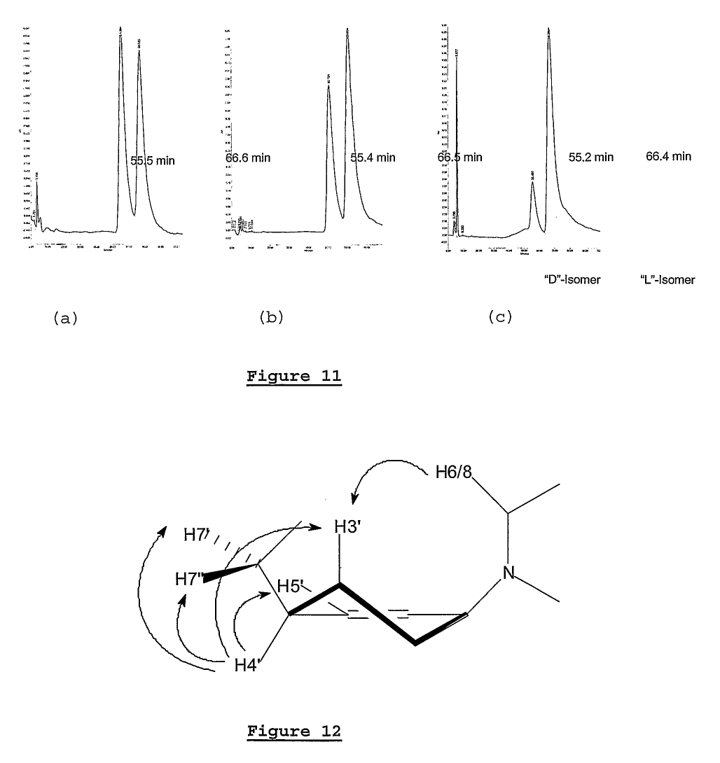

As P-glycoprotein expression leads to resistance against anti-tumor drugs, it would be important to evaluate if the inhibition of P-glycoprotein expression results in a parallel increase in cytotoxic activity of such drugs (e.g. Adriamycin) and if the inhibition of P-glycoprotein expression will result in accumulation of substrate molecules (e.g. Rhodamine 123) into the cell. These results are shown in FIGS. 6 and 7. The cells were transfected with the unmodified, single modified (GS 2186) and double modified (GS 2179 and GS 2183, also indicated as 2179/2183) siRNA. After pre-treatment with the siRNA, the cytotoxic effect of Adriamycin was measured. Pretreatment with CeNA modified siRNA leads to an increase in the cytotoxicity of Adriamycin, with the double modified siRNA being more active than the single modified siRNA, at low drug concentration. A parallel experiment measuring Rhodamine 123 uptake confirms the cytotoxicity data, i.e. that cells treated with double modified siRNA are able to accumulate more Rhodamine 123 than ones treated with unmodified or single modified siRNA (FIG. 7).

The enzymatic stability of the oligomers used in this experiment was tested. It was found that even introduction of a single CeNA unit in siRNA molecules increased the stability to both pancreatic RNase and the nucleases in serum (FIG. 8).

We investigated the influence of incorporation of CeNA nucleotides in a RNA duplex for siRNA applications, focusing on inhibition of MDR1. The first experiments have demonstrated that introduction of a single CeNA in an otherwise dsRNA duplex may significantly improve its siRNA effect in terms of reducing expression of P-glycoprotein, the MDR1 gene product. This is most pronounced when modifications are introduced in the middle sections of the duplex of either the sense or antisense strand. More interesting, however, is the additive effect of introduction of single CeNA modifications in both strands of the RNA duplex. Real time RT-PCR analysis demonstrates a parallel decrease in mRNA concentration. The introduction of CeNA modifications in the anti-MDR1 siRNA also resulted in increased changes in biological activity (Rhodamine123 uptake, drug sensitivity) that closely paralleled the effects on P-glycoprotein levels. One of the reasons for the increased biological activity might be the increased stability of the CeNA-containing duplexes to serum and cellular nucleases. Therefore, we evaluated the enzymatic stability of the modified duplexes. Even introduction of a single CeNA unit in the siRNA, increased the enzymatic stability considerably. Thus increased stability may be one aspect of the biological effectiveness of CeNA modified siRNAs. However, there may be other factors involved, and the study of the mode of action of fully modified CeNA will be the subject of further research. It thus seems that CeNA is not only well tolerated as substitute for RNA in a dsRNA duplex (for siRNA applications), but that its presence is beneficial for this biological effect.

Experiments with Hexitols, Altritols and Alkylated Altritols:

Also experiments with hexitol, altritol and alkylated altritol was performed according to the same methods as described herein. The results are shown in the figures.

Therefore, the present invention relates to the use of 6-membered ring containing nucleotides or nucleosides for the construction of oligomers to be used in RNA interference. Another aspect of the present invention relates therefore to the use of oligomers comprising at least one 6-membered ring containing nucleotide for RNA interference. Another aspect of the invention relates to compositions comprising oligomers, whereof at least a part include a 6-membered ring containing nucleoside or nucleotide. Yet another aspect of the present invention relates to a method of performing RNA interference, said method comprising exposing a double stranded polynucleotide to a target nucleic acid, wherein said double stranded polynucleotide is comprised of a sense strand and an antisense strand, and wherein at least one of said sense strand and said antisense strand comprises at least one 6-membered ring containing nucleotide.

The 6-membered ring containing nucleosides or nucleotides can be hexitol, altritol, altritol-derived, cyclohexenyl, ribo-cyclohexenyl or ribo-cyclohexenyl-derived nucleosides or nucleotides. In a preferred embodiment, the 6-membered ring containing nucleosides or nucleotides are altritols.

### Synthesis of C₂-Substituted Cyclohexenyl Nucleoside or Nucleotide Analogs and Oligomers Thereof.

The synthesis of ribo-cyclohexenyl adenosine comprised an inverse-electron-demand Diels-Alder cycloaddition reaction of 2,2-dimethyl-1,3-dioxole (dienophile) with 3-bromo-2H-pyran-2-one (diene) to construct a bicyclic intermediate 4,4-dimethyl-3,5,8-trioxa-tricyclo[5.2.2.0]undec-10-en-9-one as described in literature. Reduction and ring opening of the lactone gives a protected diol. Treating the diol with tert-butyldimethylsilyl chloride yielded a monosilylated product.

However, to obtain rCe-A the configuration of the allylic hydroxyl group has to be inverted. Therefore the allylic hydroxyl group was oxidized to the corresponding enone. Reduction of the enone with NaBH₄ in the presence of CeCl₃.7H₂O provides the α-alcohol as major component. Introduction of the base moiety onto the cyclohexenyl ring can be effected by a SN₂ reaction, following the Mitsunobu protocol. Purification of the different configuration can than be performed by using an enzymatic method.

The synthesis of the ribo-cyclohexenyl nucleosides with other heterocycles such as guanine, uracil, thymine or cytosine can be performed according to the same procedures as described for adenine herein and according to the procedures of the literature like in Gu, P. et al. Tetrahedron 2004, 60, 2111-2123.

The resolution of the different configuration of the D- and L-cyclohexenyl adenine nucleosides was performed as described herein with the use of an enzyme-catalyzed resolution strategy (adenosine deaminase). However, other methods to separate the D- and L-configurations of the cyclohexenyl nucleoside analogs could be used like for example described in literature in Wang, J., J. Org. Chem. 2001, 66, 5478-8482. The strategy uses (R)-(−)-methyl-mandelic acidin order to form diastereoisomeric esters which can than be separated by chromatographic techniques.

For synthesis of oligomers comprising the novel ribo-cyclohexenyl nucleosides, the nucleoside analogs have to be converted to their protected phosphoramidite derivatives. Introduction of the amino-protecting groups for adenine and guanine is known in the art and can be performed as described in Gu, P. et al. Tetrahedron 2004, 60, 2111-2123.

### EXAMPLES

### Example 1

### Materials and Methods Used/that can be Used

The procedures used were as described in Xu, D. et al. Mol. Pharmacol. Vol 66, 268-275, 2004 which is incorporated as reference herein.

Preparation of the Nucleosides, the Oligomers and siRNAs Duplexes

The hexitol, altritol and cyclohexenyl nucleosides/nucleotides were prepared as described previously in EP0646125, WO0218406 and EP1210347 respectively.

The phosphoramidite building blocks or derivatives of the hexitol, altritol and cyclohexenyl nucleosides/nucleotides or the oligomers HNA, ANA and CeNA were prepared according to the literature (see above) and previously reported procedures (i.e. a. De Bouvere, B., Kerremans, L., Rozenski, J., Janssen, G., Van Aerschot, A., Claes, P., Busson, R. and Herdewijn, P. (1997) Improved synthesis of anhydrohexitol building blocks for oligonucleotide synthesis. Liebigs Ann.-Rec., 1453-1461; b. DeWinter, H., Lescrinier, E., Van Aerschot, A. and Herdewijn, P. (1998) Molecular dynamics simulation to investigate differences in minor groove hydration of HNA/RNA hybrids as compared to HNA/DNA complexes. J. Am. Chem. Soc., 120, 5381-5394).

Ribo-cyclohexenyl nucleotides and oligomers were prepared as described hereunder and can be incorporated in oligonucleotides as described herein for other modified nucleotides.

RNA oligomer production was performed as known in the literature. All the oligonucleotides used in this study were purified by high-performance liquid chromatography (HPLC). Mass spectra were acquired for on a quadrupole/orthogonal-acceleration time-of-flight tandem mass spectrometer equipped with a standard electrospray ionization interface:

SiRNAs were made by forming duplexes from mixtures of the individual sense and antisense strands/oligomers. In order to form the siRNA duplex, the mixture of two oligomers (equimolar amounts) was heated briefly to 96° C. in a M J Research thermal cycler and then allowed to anneal at 25° C. An oligomer, containing 6-membered ring containing nucleotides, was mixed with an unmodified oligomer in order to form a siRNA duplex. The sense or antisense oligomer was selected from the lists in example 2. As a control an unmodified siRNA duplex was used.

### Cells

NIH 3T3 cells stably transfected with a plasmid containing the human MDR1 gene (pSKI MDR) were a gift from M. M. Gottesman (Kane, S. E., Reinhard, D. H., Fordis, C. M., Pastan, I. and Gottesman, M. M. (1989) A new vector using the human multidrug resistance gene as a selectable marker enables overexpression of foreign genes in eukaryotic cells. Gene, 84, 439-446.). The NIH 3T3 cells expressing the human MDR1 gene (NIH 3T3 MDR cells) were grown in Dulbecco's Modified Eagle's Medium (DMEM) containing 10% fetal bovine serum (FBS) and 60 ng/ml of colchicine in a humidified atmosphere of 95% air and 5% CO₂ at 37° C. MDR NCI/ADR-RES breast carcinoma cells also over-expressing the MDR1 gene were obtained from the ATCC and grown in minimum essential medium (MEM) containing 10% FBS under the same conditions. These cells have attained their MDR status via chronic exposure to doxorubicin. The multidrug resistant cell line MES-Sa/DX-5 was obtained from the ATCC. This uterine sarcoma fibroblast expresses high levels of MDR-1 mRNA and P-glycoprotein. The cells were grown in McCoy's medium containing 10% FBS and 60 ng/ml colchicines. Both cell lines were grown in a humidified atmosphere of 95% air and 5% CO₂ at 37° C.

siRNA (Modified or Not) Treatment

NIH 3T3-MDR cells were cultured in 185 mm flasks to 95% confluency and then seeded in 12 well plates at 4×10⁴ per well in 10% FBS/DMEM−H and incubated for overnight. DMEM and MEM media were used for NIH 3T3 MDR cells and NCI/ADR-RES cells, respectively, throughout the experiments. Hybridization of the siRNA was prepared in Dharmacon universal buffer by heating the solutions to 90° C. in a Perkin Elmer PCR machine then gradual cooling to 30° C. for 30 minutes. Lipofectamine 2000 (Invitrogen, 2 μg/ml) complexes of siRNA in Opti-MEM were freshly prepared according to the manufacturer's recommendations. The cells were seeded onto six-well plates in aliquots of 3×10⁵ per well in the corresponding medium containing 10% FBS. After 24 h, cells were treated with the oligonucleotide Lipofectamine 2000 complex (2 μg/ml) in the corresponding fresh medium (2 ml) containing 10% FBS for 4 h at 37° C. The cells were then washed twice with 10% FBS/DMEM or 10% FBS/MEM and incubated in the corresponding medium at 37° C. For studies of cytotoxicity, expressed P-glycoprotein levels through immunostaining (by flow cytometry and western blotting), and Rhodamine 123 accumulation (by flow cytometry), cells were further incubated in the corresponding medium containing 2% FBS for 64 h.

The compounds with cationic lipids were mixed in 10% FBS/DMEM−H and incubated with cells at 37° C. for 4 hours, media was then removed and replaced with 2% FBS/DMEM−H and incubated an addition 68-72 hours.

### Western Blotting for P-Glycoprotein Expression

After treating with an oligonucleotide as described above, and further incubation for 64 h, NIH 3T3 MDR cells can be detached, counted for normalization and harvested for western analysis. The cells can be lysed in a modified radioimmunoprecipitation buffer (150 mM NaCl, 50 mM Tris pH 7.4, 1% NP40, 0.5 mM deoxycholate, 5 mM EDTA, 1 mM dithiothreitol, 2 mM phenylmethylsulfonyl fluoride, 0.1% aprotinin and 0.1% SDS), and lysates can be microfuged at 12 000 r.p.m. for 10 min at 4° C. Equal amounts of protein (20 μg) can be mixed with 4×SDS sample buffer and can be boiled for 5 min. The proteins were electrophoresed on a 7% SDS-polyacrylamide gel and the separated proteins can be transferred onto polyvinylidene difluoride membranes (Millipore, Bedford, Mass.). The MDR1 expression can be detected using monoclonal anti-P-glycoprotein C219 antibody (Signet Laboratory, Dedham, Mass.) at 2 μg/ml in 1% BSA. Peroxidase-conjugated rat anti-mouse immunoglobulin G (IgG) antibody (Calbiochem, San Diego, Calif.) at a dilution of 1:3000 can be used as a secondary antibody in 3% BSA/1% Tween-20. Actin can be detected by anti-actin primary antibody (Sigma-Aldrich) at a dilution of 1:6000. Signals can be detected by enhanced chemiluminescence (ECL kit, Amersham Biosciences, Piscataway, N.J.).

### Cytotoxicity

NIH 3T3 MDR cells were treated with Lipofectamine 2000 complexes of siRNAs, washed and further incubated as described. The cells were then detached, washed twice with PBS and counted using an Elzone particle cell counter (Micromeritics, Norcross, Ga.) to measure the number of surviving cells.

### Immunostaining of P-Glycoprotein

The P-glycoprotein expression on viable cell membrane surfaces was studied by immunostaining using a flow cytometry assay. After treating NIH 3T3 MDR cells or NCI/ADR-RES cells with oligonucleotide and further incubating them for 64 h, as described above, the cells were trypsinized, washed twice with PBS, counted for normalization and incubated with MRK16 (Kamiya, Seattle, Wash.) anti-P-glycoprotein primary antibody in PBS (20 μg/ml, 45 min) at 4° C. The cells were then washed with PBS three times, and treated with an anti-mouse IgG secondary antibody conjugated with R-phycoerythrin (Sigma, St Louis, Mo.) for 30 min in 10% FBS/PBS at 4° C. and then washed with 10% FBS/PBS three times. The levels of immunostaining by R-phycoerythrin in viable cells (identified by light scattering) were then quantified on a Becton Dickinson flow cytometer using Cicero software (Cytomation, Fort Collins, Colo.).

### Rhodamine 123 Accumulation

The fluorophore Rhodamine 123 is a substrate for the P-glycoprotein efflux pump. Thus, the Rhodamine 123 accumulation is often used as a surrogate for drug uptake. NIH 3T3 MDR cells can be treated with siRNAs complexed with Lipofectamine 2000 as described above. After 64 h, the cells can be trypsinized and suspended in DMEM/10% FBS. The cells can than be washed once and resuspended in complete medium and warmed to 37° C. before adding Rhodamine 123 (1 μg/ml). After 1 h at 37° C., cells can be washed once with cold PBS and resuspended in PBS. The accumulation of Rhodamine 123 inside viable cells can be measured by flow cytometry as described (Alahari, S. K., Dean, N. M., Fisher, M. H., Delong, R., Manoharan, M., Tivel, K. L. and Juliano, R. L. 1996 Inhibition of expression of the multidrug resistance-associated P-glycoprotein of by phosphorothioate and 5′ cholesterol-conjugated phosphorothioate antisense oligonucleotides. Mol. Pharmacol., 50, 808-819).

RNA extraction and Real-Time RT-PCR

Total RNA was isolated using a Tri Reagent kit (Molecular Research Center, Inc), and cDNA was synthesized from total RNA using an oligo-dT primer. Primers (Oligonucleotide Synthesis Core Facility, University of North Carolina) and probes (Integrated DNA Technologies, Santa Clara, Calif.) were designed using Primer 4 software and were designed to span exon-intron junctions. MDR1 probes were labeled at the 5′ end with the reporter dye 5-carboxyfluorescein and at the 3′ end with the quencher dye 5-carboxytetramethylrhodamine. The human glyceraldehyde 3-phosphate dehydrogenase (GAPDH) probe was labeled at the 5′ end with the reporter dye tetrachloro-6-carboxy-fluorescein and at the 3′ end with the quencher dye 5-carboxytetramethylrhodamine. The sequences are as follows: for MDR1: probe, 5′-TCAGTAGCGATCTTCCCAGCACCT-3′ (SEQ ID NO: 1); sense primer, 5′-GTCTGGACAAGCACTGAAA-31 (SEQ ID NO: 2); antisense primer, 5′-AACAACGGTTCGGAAGTTT-3′(SEQ ID NO: 3). For human GAPDH, probe, 5′-CAAGCTTCCCGTTCTCAGCC-3′ (SEQ ID NO: 4); sense primer, 5′-ACCTCAACTACATGGTTTAC-3′ (SEQ ID NO: 5); antisense primer, 5′-GAAGATGGTGATGGGATTTC-3′ (SEQ ID NO: 6). PCR reactions of cDNA samples and standards were performed with the use of Platinum Quantitative PCR SuperMix-UDG (Invitrogen) in a total reaction volume of 15 μl. Real-time PCR was performed using the ABI PRISM 7900 sequence detection system (Applied Biosystems, Foster City, Calif.). The PCR conditions were 50° C. for 2 min, 95° C. for 2 min, followed by 40 cycles of 95° C. for 15 s and 56° C. for 1.5 min. Standard curves were constructed with PCR-II TOPO cloning vector (Invitrogen) containing the same fragment as amplified by the Taqman system. The expression in each sample was calculated based on standard curves generated for MDR1 or GAPDH. Samples were normalized by dividing the copies of MDR1 by the copies of human GAPDH.

### Nuclease Stability

For nuclease stability experiments, unmodified or CeNA modified siRNA duplexes were incubated either with pancreatic RNase or with 10% FES. Thereafter the material was analyzed on 3% agarose/ethidium bromide gels in BPB/XC loading buffer and electrophoresed at 100 volts for 45 minutes and residual duplexes imaged by ultraviolet illumination.

### Example 2

### siRNA Design and Sequences

The siRNA duplexes with sequence 5′-GUA DTG ACA GCU AUI CGA ATT-3′ (SEQ ID NO:7) is the sense strand and were designed to target the coding region at nt 1545-1565 of MDR1 mRNA (ORF1). The sequence 5′-UUC GAA UAG CUG UCA AUA CTT-3′ is the antisense strand.

a) Oligomers comprising cyclohexenyl containing nucleotides (CeNA)

(SEQ ID NOs:)

| | |
|---|---|
| GS 2176 | |
| 5′-G*UA UUG ACA GCU AUU CGA ATT-3′ | (8) |
| GS 2177 | |
| 5′-GUA UUG* ACA GCU AUU CGA ATT-3′ | (9) |
| GS 2178 | |
| 5′-GUA UUG ACA G*CU AUU CGA ATT-3′ | (10) |
| GS 2179 | |
| 5′-GUA UUG ACA GCU AUU CG*A ATT-3′ | (11) |
| GS 2180 | |
| 5′-GUA* UUG A*CA GCU AUU CGA ATT-3′ | (12) |
| GS 2181 | |
| 5′-GUA UUG ACA GCU AUU CGA* ATT-3′ | (13) |
| GS 2182 | |
| 5′-G*UA* UUG ACA GCU AUU CGA ATT-3′ | (14) |
| GS 2183 | |
| 5′-UUC GAA UAG CUG* UCA AUA CTT-3′ | (15) |
| GS 2184 | |
| 5′-UUC GAA UAG CUG* UCA AUA* CTT-3′ | (16) |
| GS 2185 | |
| 5′-UUC GAA UAG CUG UCA AUA* CTT-3′ | (17) |
| GS 2186 | |
| 5′-UUC GAA UAG CUG UCA A*UA CTT-3′ | (18) |

b) oligomers comprising hexitol containing nucleotides (HNA)

(SEQ ID NOs:)

| | |
|---|---|
| GS 2187 | |
| 5′-GUA* UUG A*CA GCU AUU CGA ATT-3′ | (19) |
| GS 2188 | |
| 5′-GUA UUG ACA GCU AUU CGA* ATT-3′ | (20) |
| GS 2189 | |
| 5′-UUC GAA UAG CUG UCA AUA* CTT-3′ | (21) |
| GS 2190 | |
| 5′-UUC GAA UAG CUG UCA A*UA CTT-3′ | (22) |

c) oligomers comprising altritol containing nucleotides (ANA)

(SEQ ID NOs:)

| | |
|---|---|
| GS 2191 | |
| 5′-GUA* UUG A*CA GCU AUU CGA ATT-3′ | (23) |
| GS 2192 | |
| 5′GUA UUG ACA GCU AUU CGA* ATT-3′ | (24) |
| GS 2193 | |
| 5′UUC GAA UAG CUG UCA AUA* CTT-3′ | (25) |
| GS 2194 | |
| 5′UUC GAA UAG CUG UCA A*UA CTT-3′ | (26) |

d) oligomers comprising alkylated altritol containing nucleotides, namely 3-OMe HNA (ANA-Alk)

(SEQ ID NOs:)

| | |
|---|---|
| GS 2286 | |
| 5′-GUA UUG ACA GCU AU*U* C*GA ATT-3′ | (27) |
| GS 2287 | |
| 5′-GUA U*U*G AC*A GC*U* AU*U* C*GA | (28) |
| ATT-3′ | |
| GS 2288 | |
| 5′-GUA U*UG AC*A GCU AUU CGA ATT-3′ | (29) |
| GS 2291 | |
| 5′-UUC GAA UAG CUG UCA AUA C*TT-3′ | (30) |

The modified nucleotides (6-membered ring containing nucleotides) in the oligomers are indicated with * after the modified nucleotide starting from the 5′-end. As shown herein above, modified nucleotides were present in the sense and the antisense oligomers. The “T” at the 3′ end, indicated in bold, represent the 3′ overhang.

### Example 3

### Results of siRNA Treatment with siRNA Duplexes Wherein Only One Oligonucleotide of the Duplex Comprises Modified Nucleotides

In the experiments performed with the siRNA duplexes the siRNAs with modified nucleotides showed a higher activity (reduction of Pgp) than the control siRNA (unmodified RNA). Especially the altritol containing siRNAs were highly active.

As an example, hereunder are the results of a Pgp reduction experiment, as performed as described above, wherein siRNA duplexes were used (50 nM) and the results were measured 4 hours after transfection (Table 1).

TABLE 1

| | | | % P-gp |
|---|---|---|---|
| | % Cell | % P-gp | reduction - |
| | Toxicity | reduction | duplex Control |
| Duplex control | | 39 | **** |
| CeNA | | | |
| GS 2177 (sense) | 0 | 41 | 2 |
| GS 2178 (sense) | 0 | 46 | 7 |
| GS 2179 (sense) | 0 | 55 | 16 |
| GS 2180 (sense) | 0 | 40 | 1 |
| GS 2181 (sense) | 0 | 50 | 11 |
| GS 2182 (sense) | 0 | 46 | 7 |
| HNA | | | |
| GS 2187 (sense) | 6 | 46 | 7 |
| GS 2188 (sense) | 0 | 50 | 11 |
| GS 2189 (antisense) | 0 | 56 | 17 |
| ANA | | | |
| GS 2191 (sense) | 0 | 55 | 16 |
| GS 2192 (sense) | 0 | 48 | 9 |

(sense): modification in the sense strand
(antisense): modification in the antisense strand

Of another experiment under the same conditions as hereinabove, the results are shown hereunder (Table 2). One of the ANA containing duplexes shows practically a 100% increase in P-gp reduction.

TABLE 2

| siRNA | | % P-gp reduction - |
|---|---|---|
| Oligo | % P-gp reduction | duplex Control |
| CeNA | | |
| GS2183 | 51 | 19 |
| ANA | | |
| GS2193 | 60 | 28 |
| GS2194 | 55 | 23 |
| HNA | | |
| GS2189 | 53 | 21 |
| GS2190 | 45 | 13 |
| siRNA | 32 | ****** |
| Control | | |

The results of another experiment are shown hereunder (Table 3).

TABLE 3

| siRNA | | % P-gp reduction - |
|---|---|---|
| Oligo | % P-gp reduction | duplex Control |
| ANA-Alk | | |
| GS2286 | 52 | 7 |
| GS2291 | 62 | 17 |
| siRNA | 45 | ****** |
| Control | | |

More results are shown in the figures.

### Example 5

### Results of siRNA treatment with siRNA Duplexes Wherein Both Oligonucleotides of the Duplex Comprise Modified Nucleotides

The synthesis and experiments with siRNA duplexes with modifications in both oligomers are performed as described herein. The results are shown in the figures, namely FIG. 3, FIG. 4, FIG. 5, FIG. 6, FIG. 7 and FIG. 8.

The results show that inhibition of P-gp reduction is very high for most of the siRNA duplexes with modifications in both oligomers and is higher than for the siRNA duplexes with modified nucleotides in only one strand.

### Example 6

### Synthesis of Ribo-Cycleohexenyl Nucleosides and Nucleotides and Oligomers Containing Said Nucleotides

The synthesis of ribo-cyclohexenyl nucleic acids is exemplified by the synthesis of the adenine containing ribo-cyclohexenyl.

### Results and Discussion

Key step of the synthesis of ribo-cyclohexenyl adenosine 18 is an inverse-electron-demand Diels-Alder cycloaddition reaction (Posner G. H. et al. Tetrahedron 1990, 46 (13), 4573-4586; Posner G. H. et al. Tetrahedron Letters 1991, 32 (39), 5295-5298; Posner G. H. et al. J. Org. Chem., 1991, 56, 4339-4341) of 2,2-dimethyl-1,3-dioxole (dienophile) 6 with 3-bromo-2H-pyran-2-one (diene) 10a to construct a bicyclic intermediate 11. 2,2-Dimethyl-1,3-dioxole 6 can be obtained via a cascade of Diels-Alder (DA) and Retro-Diels-Alder reactions (RDA) (Posner G. H. et al. Tetrahedron 1990, 46 (13), 4573-4586; Organic Syntheses, an improved preparation of 3-bromo-2H-pyran-2-one, p112-116) outlined in Scheme 1.

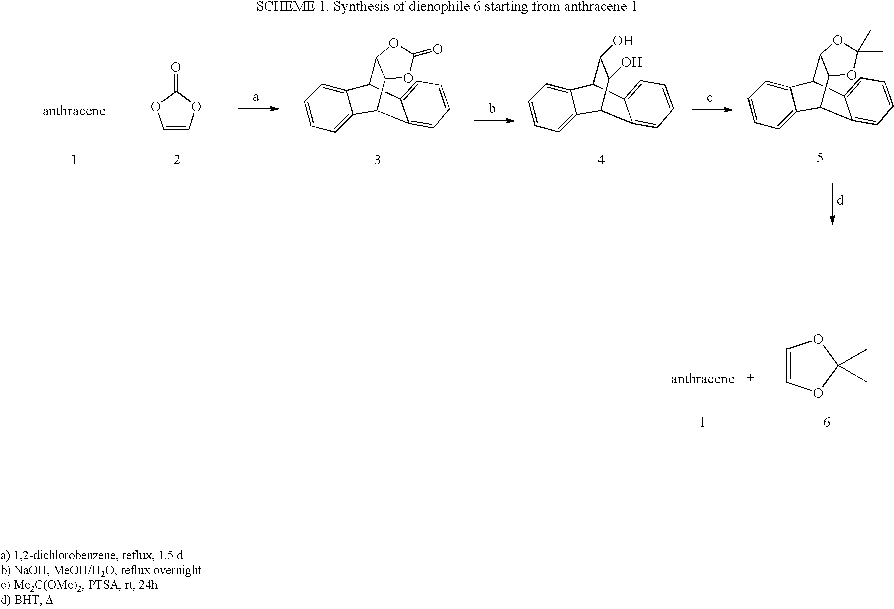

Diels-Alder reaction of anthracene 1 and vinylene carbonate 2 provides 3 in high yield (94%). Hydrolysis of 3 with NaOH in MeOH gives rise to the diol 4 (76%). To obtain 6, diol 4 is first converted into the acetal 5 (96%), using 2,2-dimethoxypropane/p-toluenesulfonic acid at rt. Thermally cracking of 5 leads to 55% of 6 by Retro-Diels Alder reaction (RDA). Diene 10a is obtained by a sequence of selective bromination reactions, followed by elimination as outlined in Scheme 2.

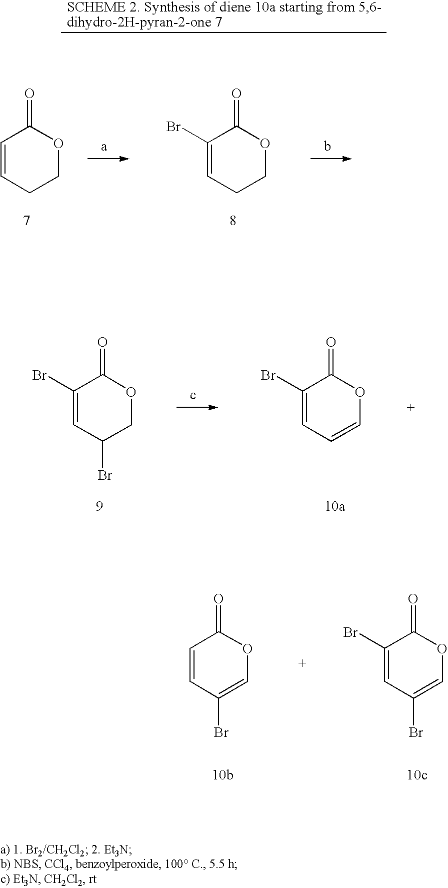

Selective bromination in position 3 of 5,6-dihydro-2H-pyran-2-one 7 in CH₂Cl₂ gives 3-bromo-5,6-dihydro-2H-pyran-2-one 8 (82%). A second bromination of 8 in allylic position was carried out with N-bromosuccinimide (NBS) to obtain 9 (89%). Subsequent elimination with Et3N yields 3-bromo-2H-pyran-2-one 10a (43%). The formation of the major by-product, 5-bromo-2H-pyran-2-one 10b, results from prototrophic migration in basic medium followed by elimination of HBr.

The key step Diels-Alder reaction was carried out by heating 10a and 6 together with a small amount of ethyldiisopropylamine in a sealed pressure tube at 90° C. for 4 days. Replacement of the bridgehead bromine by hydrogen using tributyltin hydride and AIBN (radical mechanism) provides the halogen-free bicyclic lactone 12. Reduction and ring opening of the lactone 12 with lithium aluminium hydride (Roberts S. M. et al. J. Chem. Soc. Perkin. Trans. 1, 1995, 12, 1499-1504) gives diol 13 in good yield (86%). Treating 13 with 1.2 equivalents of tert-butyldimethylsilyl chloride in DMF in presence of 1.5 equivalents of imidazole at 0° C. allows protection of the primary hydroxyl group. Monosilylated 14 was obtained in 59% yield, together with 17% of the starting material 13 was recuperated.

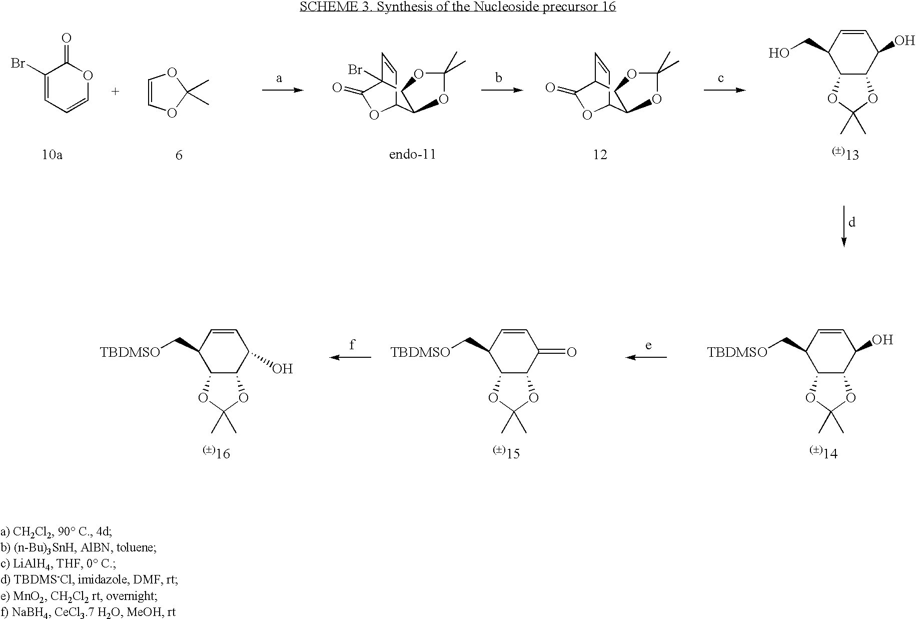

Cycloadduct 11 was formed in a 4:1 mixture of endo:exo isomers. Structural proof for the endo isomer could be achieved with the help of NOE-difference spectroscopy: Irradiation of H5/6 caused positive NOE enhancement (2.53%) of CH3 a/b, whereas irradiation of CH3 a/b led to positive NOE of 1.6% (H2/3) and 0.5% (H5/6), respectively (FIG. 2).

To obtain rCe-A (18) the configuration of the allylic hydroxyl group has to be inverted. Therefore the allylic hydroxyl group 14 was oxidized to the corresponding enone 15 by using manganese dioxide in CH₂Cl₂ (84%). Reduction of the enone 15 with NaBH₄ in the presence of CeCl₃.7H₂O provides the α-alcohol 16 (72%). A small amount of the β-alcohol 14 (9%) was also found. Introduction of the base moiety onto the cyclohexenyl ring can be effected by a SN₂ reaction, following the Mitsunobu protocol (Scheme 4).

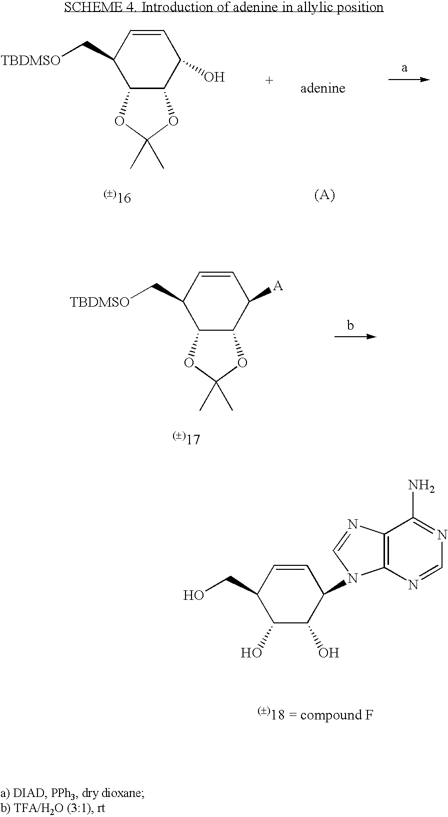

Treatment of 16 with adenine in the presence of PPh₃ and DIAD in dry dioxane at room temperature gives rise to 17 (62%). Complete deprotection of 17 with TFA/H₂O (3:1) at room temperature overnight affords the adenine compound (±) 18 as racemic mixture. The potential of this nucleoside analogue to mimic adenosine in biological systems is tested by its substrate specificity for an adenosine metabolic enzyme, i.e. adenosine deaminase. (Adenosine Deaminase (EC 3.5.4.4) from calf intestinal mucosa, purchased from Sigma—Aldrich; Product No. A-1030, type VIII. One unit of this enzyme preparation will delaminate 1.0 μmol of adenosine to inosine per minute at pH 7.5 at 25° C.). Likewise, this enzyme may be used to resolve the obtained racemic mixture of “ribo” cyclohexenyl-A (±18) (Secrist, J. et al. J. Med Chem, 1987, 30, 746-749). We may conclude that a resolution of the two enantiomers of (±)-rCe-A 18, with concomitant conversion of the D-like enantiomer into an inosine analogue, is possible by selective enzymatic deamination reaction of (±) 18 using adenosine deaminase (ADA) (Sandaralingam, M. in Ann. New York Acad. Sci., 1975, Vol. 255, 3-42 (ed. A. Bloch) (Scheme 5).

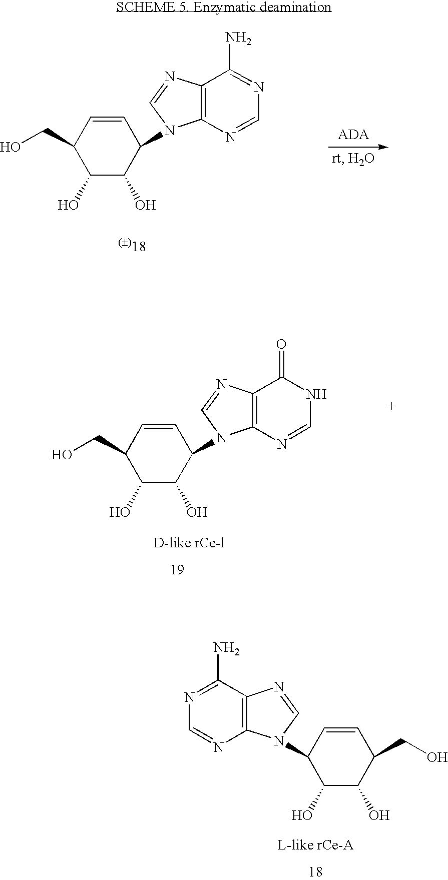

Racemic 18 dissolved in ethanol, is well resolved using a Chiralpak column and hexane/EtOH 85:15 as eluent. After treatment of 18 with ADA a progressive disappearance of the first peak is observed after 12 hrs and after 24 hrs of incubation, whereas the second peak remains unaffected. Therefore the HPLC analyses indicates the less mobile enantiomer (retention time ˜55 min) being a substrate for ADA which is an indication that this isomer corresponds to a D-like nucleosides. The more mobile one (retention time ˜66 min) resembling a L-nucleoside. The formation of the polar inosine analog 19 could be demonstrated by TLC separation and mass spectrometry indicating that ADA transferred one enantiomer of (±) 18 into the inosine-analogue 19 (FAB⁺m/z C₁₂H₁₄N₄O₄=278.1015).

The synthesis of the guanine C₂-substituted cyclohexenyl nucleoside analog can be performed by adding 2-amino-6-chloropurine to compound 16 under Mitsunobu condensation reaction conditions as described for adenine for the synthesis of 17. After a separation of the N₇- and the N₉-isomer, the desired guanine N₉-derivative can be obtained by treating the compound with TFA-H₂O (3:1) at room temperature overnight.

For coupling of thymine to 16, the Mitsunobu condensation reaction can be used. The cytosine analog can be obtained starting from the uracil congener. Uracil can be introduced by reacting uracil and NaH with 16. Deprotection with TFA/H₂O yields the uracil C₂-substituted-cyclohexenyl nucleoside analog. Modifying uracil into cytosine on the hydroxy-protected cyclohexenyl nucleoside can be performed by using POCl₃, 1,2,4-triazole, NEt₃ in MeCN, followed by treatment with NH₄OH in dioxane.

For synthesis of oligomers the following steps can be followed:

For adenine, compound 17 can be protected with a benzoyl protecting group after which the hydroxy-protecting groups are cleaved with 80% TFA/H₂O solution. For guanine, deprotection with TFA/H₂O can first be performed, followed by protection of the exocyclic amino group with the isobutyryl group via a transient protection approach as described in the prior art. Monomethoxytritilation in pyridine of the primary hydroxyl group is the following step for the adenine and guanine nucleoside analogs and can be performed with MMTrCl in pyridine. Separation by HPLC of the mono- or di-titillated products or influencing the reaction conditions like lowering the temperature can be necessary. Subsequently, the secondary hydroxyl groups are further reacted with 2-cyanoethyl-N,N-diisopropylchlorophosphoramidite together with DIEA in DCM to yield the desired C₁-phosphoramidite after HPLC separation.

However, an alternative method which would include less difficult separation steps would be possible. Exemplified with as base adenine, the method would comprise the following steps starting from the (chirally pure) exocyclic amino-benzoyl protected 18:

selective protection of the C₁—OH and C₆₋hydroxymethyl with a benzylidene through reaction with 1.05 eq. freshly dried ZnCl₂ and 5 eq. benzaldehyde during two days;

protection of the C₂—OH with TBDMS (by reacting with TBDMSCl, imidazole in DMF) or benzoyl (by reacting with benzoylCl, DMAP in pyridine) protecting groups;

deprotection of the bezilidene with a 90% TFA solution.

monomethoxytritilation of the primary alcohol as described in the prior art, preferably at 0° C. so that the secondary C₁—OH does not react;

introduction of the phosphoramidite by using 2-cyanoethyl-N,N-diisopropylchlorophosphoramidite as described in the prior art.

For the cytosine C₂-substituted cyclohexenyl nucleoside analog, a benzoyl protection of the exocyclic amino group can be used. Therefore, benzylchloride is added to the cytosine analog with the hydroxy groups still protected in pyridine. Subsequently, for the cytosine, uracil and thymine C₂-substituted cyclohexenyl nucleoside analog, the same procedure as described for adenine and guanine can be applied to obtain the protected phosphoramidite nucleoside analogs.

### Experimental Section

NMR spectra were recorded on a Varian, Gemini 200 spectrometer (1H, 200 MHz, 13 C, 50 MHz) and a Varian Unity 500 spectrometer (1H-500 MHz, 13 C, 125 MHz). 13 C and 1H are referred to TMS. All NH/OH protons were assigned by exchange with D2O. Exact mass measurements were performed on a quadrupole-time of flight mass spectrometer (Q-Tof-2, Micromass, Manchester, UK) equipped with a standard electrospray ionisation (ESI) interface. Samples were infused in a methanol:water (1:1) mixture at 3 μl/min. Precoated Alugram SIL G/UV254 plates were used for TLC and spots were examined with UV light, KMnO4 spray and Ce(SO4)2/(NH4) 6MoO4 spray and silica (200-425 mesh) was used for column chromatography. Melting points (mp[° C.]) were determined with a Büchi-SMP-20 capillary melting apparatus. All air-sensitive reactions were carried out under nitrogen. THF, toluene, 1,4-dioxane were distilled from sodium/benzophenone, and CH2Cl2 from P2O5. Enantiomer compositions were determined by chiral HPLC analysis with Chiralpak AD column (250×4.6 mm) on a Waters 6000 controller liquid chromatograph equipped with a Waters 2487 UV detector. Elementary Analysis was obtained from the “Microanalytical Labor”, Fakultät für Chemie, Universität Konstanz.

The starting products of the key step of this synthesis, the RDA-reaction, anthracene-2,2-dimethyl-1,3-dioxole adduct (5) and 3-bromo-2H-pyran-2-one (10) were prepared according to literature.

### 2,2-Dimethyl-1,3-dioxole (6)

(Posner G. H. et al. Tetrahedron 1990, 46 (13), 4573-4586; Field N.D. J. Am. Chem. Soc. 1961, 83, 3504-5307.)

Before starting the reaction, all glasswork was dried overnight in an oven at 80° C. The starting material (21.0 g, 0.075 mol of 5 and a few crystals of BHT) was placed in a 100 ml flask and put on the lyofilisator during 24 hrs to be sure that all water has been removed. The whole apparatus (see picture) was flushed 3 times with nitrogen. The RDA-reaction is carried out under N2-protection. The temperature of the collecting tube has been adjusted to ±−50° C. with acetone/dry ice. (Dioxole 6 is an easy volatile liquid, solidifying at ±−70° C.). After melting the solid with a heat-gun, and increasing the temperature to about 600° C. the RDA reaction started, indicated by vigorous boiling. The formed dioxole 6 was collected in the pre-cooled tube; heating was continued till no more product distilled (1.5 hrs). Dioxole 6 (4.01 g, 55%) was collected as colourless liquid and was stored at −20° C. The identity of 6 was proven by means of NMR spectroscopy. 1H NMR (CDCl3) δ 1.52 (s, 6H, 2 CH3), 6.17 (s, 2H, CH); 13C NMR (CDCl3) δ 24.8 (CH3), 114.1 (C(CH3)₂), 126.6 (2×CH);

### 1-Bromo-4,4-dimethyl-3,5,8-trioxa-tricyclo[5.2.2.0]undec-10-en-9-one (11)

(Organic Syntheses, an improved preparation of 3-bromo-2H-pyran-2-one, p112-116; Field N. D. J. Am. Chem. Soc. 1961, 83, 3504-5307; Roberts S. M.; Sutton P. W., J. Chem. Soc. Perkin. Trans. 1, 1995, 12, 1499-1504). A 15 mL pressure tube (Aldrich) was charged with 10a (1.28 g, 7.31 mmol), 6 (3.56 g, 35.59 mmol, 4.87 eq) and Hünig's base (EtN(i-Pr)2, 85.3 mg 0.66 mmol, 0.09 eq). After adding CH2Cl2 (pa, 5.6 mL) the tube was sealed and placed in an oven (90° C.) for 4 days. After cooling to rt, the resulting brown-yellow solution was concentrated. The residue was purified by flash chromatography on silica gel (50 g SiO2, the column was packed with hexane-EtOAc (10:1+1% Et3N) and eluted with hexane-EtOAc (1:1+1% Et3N) in less then 3 min due to instability of 11 on the silica gel). The resulting yellow solution was concentrated to give an orange-yellow oil as a mixture of the endo- and exo-isomer. The spectroscopic data of the endo isomer are given. Rf 0.68 (hexane-EtOAc 2:1). 1H NMR (CDCl3) δ 1.38 (s, 3H, CH3), 1.42 (s, 3H, CH3), 4.63 (dd, 1H, J=6.9 Hz; 1.2 Hz, H3), 4.77 (dd, 1H, J=7.0 Hz; 4.4 Hz, H2), 5.27 (td, 1H, J=4.5 Hz; 2.2 Hz, H1), 6.34-6.48 (m, 2H, H5; H6) 13 C NMR (CDCl₃) δ 25.3 (CH3), 25.4 (CH3), 60.4 (C—Br), 73.2 (C2H), 76.7 (C1H), 79.4 (C3H), 114.5 (CMe2), 129.1 (C5H), 135.8 (C6H), 166.0 (C═O).

### 4,4-Dimethyl-3,5,8-trioxa-tricyclo[5.2.2.0]undec-10-en-9-one (12)

(Posner G. H. et al. Tetrahedron Letters 1991, 32 (39), 5295-5298).

A solution of 11 (1.34 g, 4.87 mmol), tributyltin hydride ((n-Bu)₃SnH, 1.94 mL, 7.31 mmol, 1.5 eq) and AIBN (0.28 g, 0.49 mol, 0.1 eq) in dry toluene (32 mL) was degassed under nitrogen. The solution was immersed in a preheated bath (130° C.) and refluxed for 1 h. The reaction mixture was cooled and concentrated. The residue was purified on a silica column. The column was eluted with hexane (500 mL) to remove (n-Bu)₃SnH, hexane-Et₂O 1:1 (500 mL) and hexane-Et₂O 1:2 (600 mL) to afford 12 (690 mg, 72.25%). Rf 0.62 (hexane-EtOAc 2:1) ¹H NMR (CDCl₃) δ 1.33 (s, 3H, CH₃), 1.34 (s, 3H, CH₃), 3.87 (ddd, 1H, J=1.8 Hz; 4.0 Hz; 7.4 Hz, H4), 4.60 (dd, 1H, J=6.9 Hz; 3.8 Hz, H3), 4.70 (dd, 1H, J=6.8 Hz; 4.0 Hz, H2), 5.26 (td, 1H, J=4.4 Hz; 2.2 Hz, H1), 6.40-6.51 (m, 2H, H5, H6); ¹³C NMR (CDCl₃) δ 25.3 (CH₃), 25.4 (CH₃), 46.4 (C4H), 72.5 (C1H), 75.1 (C3H), 75.8 (C2H), 113.6 (CMe₂), 129.6 (C5H), 130.3 (C6H) 170.4 (C═O).

### (±)-(3aS,4R,7R,7aR)-7-(Hydroxymethyl)-2,2-dimethyl-3a,4,7,7a-tetrahydro-1,3-benzodioxo-4-ol (13)

(Roberts S. M. et al. J. Chem. Soc. Perkin. Trans. 1, 1995, 12, 1499-1504).

To a mixture of LiAlH₄ (119 mg, 3.13 mmol, 1.5 eq) in dry THF (20 mL) at 0° C. was added a solution of 12 (410 mg, 2.09 mmol) in THF (8 mL) slowly. The reaction mixture was stirred at 0° C. for additional 15 min and at rt overnight. A saturated sodium bisulphite solution was added drop wise to the reaction mixture until a precipitate was formed. 3 mL EtOAc was added and stirred for additional 0.5 h. The precipitate was filtered and the filtrate was concentrated. The residue was purified with a silica column (the column was eluted with hexane-EtOAc 1:1) to give 13 (360 mg, 86%). R_{f} 0.15 (hexane-EtOAc 1:1); ¹H NMR (CDCl₃) δ 1.37 (s, 3H, CH₃), 1.47 (s, 3H, CH₃), 2.40 (m, 2H, CHOH, H4), 3.05 (br-s, 1H, CH₂OH), 3.78 (m, 2H, CH₂OH), 4.10-4.52 (m, 3H, H1; H2; H3), 5.68 (ddd, 1H, J=9.85 Hz; 3.7 Hz; 2.2 Hz, H5), 5.97 (dt, 1H, J=9.6 Hz; 2.7 Hz, H6); ¹³C NMR (CDCl₃) δ 24.7 (CH₃), 27.1 (CH₃), 42.3 (C4H), 64.0 (CH2), 69.7 (C1H), 75.3 (C3H), 80.5 (C2H), 108.7 (CMe₂), 127.2 (C5H), 131.8 (C6H); FAB⁺ 223.1 (M+Na)⁺; HRMS calculated for C₁₀H₁₆O₄ (M+H)⁺ 223.0946, Found 223.0949; Anal. Calc. (C₁₀H₁₆O₆): Calcd. C, 59.98, H, 8.05, Found: C, 59.48, H, 7.93.

### (±)-(3aS,4R,7R,7aR)-7-({Tert-butyl(dimethyl)silyl]oxy}methyl)-2,2-dimethyl-3a,4,7,7a-tetrahydro-1,3-benzodioxo-4-ol (14)

To a solution of 13 (620 mg, 3.09 mmol) in dry DMF (13 mL) at 0° C. imidazole (316 mg, 4.64 mmol, 1.5 eq) was added, followed by TBDMSCl (560 mg, 3.72 mmol, 1.2 eq) in 3 portions (after 0.5 h). The reaction was stirred at 0° C. for 10 min and at rt overnight and quenched with water. The resulting mixture was evaporated to remove DMF. The residue was absorbed on silica and chromatographed (hexane-EtOAc 10:1, 5:1, 1:1, 1:2) to yield 14(580 mg, 59.6%) as an oil and 13 (110 mg, 17.7%) as an oil. Spectroscopic data of 14 are given. R_{f} 0.57 (hexane-EtOAc 2:1) ¹H NMR (CDCl₃) δ 0.084 (s, 6H, Si(CH₃)₂), 0.91 (s, 9H, C(CH₃)₃), 1.36 (s, 3H, CH₃), 1.45 (s, 3H, CH₃), 2.37 (m, 1H, H4), 2.60 (br-s, 1H, OH), 3.78 (d, 2H, J=1.8 Hz, CH₂OTBDMS), 4.05-4.21 (m, 3H, H2; H3; H7), 5.73 (ddd, 1H, J=9.8 Hz; 3.5 Hz; 2.0 Hz, H5), 5.93 (dt, 1H, J=10.4 Hz; 2.6 Hz, H6); ¹³C NMR (CDCl₃) δ −5.55 (Si(CH₃)₂), 18.3 (C(CH₃)₃), 24.7 (CH₃), 25.9 (C(CH₃)₃), 27.2 (CH₃), 42.9 (C1H), 64.1 (CH₂OTBDMS), 69.5 (CHOH), 74.1 (C3H), 80.3 (C2H), 108.4 (CMe₂), 128.3 (C5H), 130.8 (C6H); FAB⁺ 337.2 (M+Na)⁺; HRMS calculated for C₁₆H₃₀O₄Si (M+Na)⁺ 337.1811, Found 337.1807; Anal. Calc. (C₁₆H₃₀O₄Si): Calcd. C, 61.11, H9.61, Found: C60.69, H, 9.19.

### (±)-(3aS,4R,7R,7aR)-7-({Tert-butyl(dimethyl)silyl]oxy}methyl)-2,2-dimethyl-7,7a-dihydro-1,3-benzodioxo-4(3aH)-one (15)

A mixture of 14 (120 mg, 0.38 mmol) and activated MnO₂ (332 mg, 3.82 mmol, 10 eq) in dry CH₂Cl₂ (6 mL) was stirred vigorously. After 12 hrs MnO₂ (332 mg, 3.82 mmol, 10 eq) was added to the mixture. After another 12 hrs the completion of the reaction was checked by TLC. Again MnO₂ (66 mg, 0.76 mmol, 2 eq) was added and stirring was continued overnight. The reaction mixture was diluted with CH₂Cl₂ and filtered through Celite and concentrated. The residue was chromatographed on silica gel (hexane-EtOAc 6:1) to give 15 (100 mg, 84%) as a white solid. R_{f} 0.68 (hexane-EtOAc 2:1); ¹H NMR (CDCl₃) δ 0.00 (s, 3H, SiCH₃), 0.03 (s, 3H, SiCH₃), 0.83 (s, 9H, C(CH₃)₃), 1.35 (s, 3H, CCH₃), 1.40 (s, 3H, CCH₃), 2.99 (m, 1H, H4), 3.74 (dd, 1H, J=10.1 Hz; 3.0 Hz, CH₂ₐOTBDMS), 3.92 (dd, 1H, J=10.0 Hz; 4.0 Hz, CH_{2b}OTBDMS), 4.35 (d, 1H, J=5.0 Hz, H2), 4.51 (dt, 1H, J=3.6 Hz; 1.65 Hz, H3), 6.19 (d, 1H, J=10.2 Hz, H6), 6.74 (dddd, 1H, J=10.3 Hz; 5.2 Hz; 1.8 Hz, H5); ¹³C NMR (CDCl₃) δ −5.7 (Si(CH₃)₂), 18.1 (SiCMe₃), 25.7 (SiC(CH₃)₃), 25.8 ((O)₂CCH₃ₐ), 27.4 ((O)₂CCH_{3b}), 41.6 (C4H), 63.3 (CH₂OTBDMS), 75.7 (C3H), 77.0 (C2H), 108.5 ((O)₂CMe₃), 129.8 (C6H), 147.2 (C5H), 196.1 (C═O); FAB⁺?(M+Na)⁺; HRMS calculated for C₁₆H₂₈O₄Si (M+Na)⁺?, Found Anal. Calc. (C₁₆H₂₈O₄Si): Calcd. C, 61.50, H, 9.03, Found: C, 61.52, H, 8.57.

### (±)-(3aS,4S,7R,7aR)-7-({Tert-butyl(dimethyl)silyl]oxy}methyl)-2,2-dimethyl-3a,4,7,7a-tetrahydro-1,3-benzodioxo-4-ol (16)

To a solution of 15 (110 mg, 0.35 mmol) in MeOH (6 mL) at rt was added CeCl₃.7H₂O (197 mg, 0.53 mmol, 1.5 eq). The mixture was stirred for 1 h, and a clear solution was obtained. NaBH₄ (16 mg, 0.42 mmol, 1.2 eq) was added in portions and H₂ evolved. The reaction mixture was stirred for 2 hrs and quenched with crushed ice. The mixture was stirred for 0.5 h and concentrated. The residue was diluted with EtOAc (15 mL), washed with H₂O and brine, dried over Na₂SO₄, and concentrated. The residue was chromatographed on silica gel (hexane-EtOAc 5:1) to give 16 (80 mg, 72.3%) as a colourless oil and 14 (10 mg, 9.1%). R_{f} 0.63 (hexane-EtOAc 2:1); ¹H NMR (CDCl₃) δ 0.05 (s, 6H, Si(CH₃)₂), 0.89 (t, 9H, J=3.0 Hz, C(CH₃)₃), 1.39 (s, 3H, CH₃), 1.45 (s, 3H, CH₃), 2.60 (m, 2H, H4, OH), 3.61 (dd, 1H, J=10.3 Hz; 4.7 Hz, CH₂ₐOTBDMS), 3.69 (dd, 1H, J=10.2 Hz; 5.0 Hz, CH_{2b}OTBDMS), 4.33-4.44 (m, 3H, H1, H2, H3), 5.82 (dd, 1H, J=10.9 Hz; 4.5 Hz, H5), 5.92 (dd, 1H, J=11.5 Hz; 2.6 Hz, H6); ¹³C NMR (CDCl₃) δ −5.6 (Si(CH₃)₂), 18.3 (C(CH₃)₃), 24.5 (CH₃), 25.8 (C(CH₃)₃), 26.4 (CH₃), 42.0 (C4H), 64.0 (CH₂OTBDMS), 64.6 (CHOH), 74.3 (C2H), 75.7 (C3H), 108.5 ((O)₂CMe₂), 129.4 (C5H), 131.1 (C6H); FAB⁺ 337.2 (M+Na)⁺; HRMS calculated for C₁₆H₃₀O₄Si (M+Na)⁺ 337.1811, Found 337.1807.

### (±)-9-[(3aS,4R,7R,7aR)-7-({[Tert-butyl(dimethyl)silyl]oxy}methyl)-2,2-dimethyl-3a,4,7,7a-tetrahydro-1,3-benzodioxol-4-yl]-9H-purin-6-amine (17)

To a mixture of 16 (220 mg, 0.70 mmol), adenine (189 mg, 1.40 mmol, 2 eq) and PPh₃ (367 mg, 1.40 mmol, 2 eq) in dry dioxane (11 mL) under N₂ at room temperature DIAD (278 μl, 1.40 mmol) was added very slowly. The reaction mixture was stirred at room temperature overnight and concentrated. The resulting residue was chromatographed on silica gel (CH₂Cl₂-MeOH, 98:2) to yield 18 (170 mg, 62.28%) as a white solid. R_{f} 0.12 (CH₂Cl₂:MeOH 98:2), ¹H NMR (CDCl₃) δ 0.10 (s, 6H, Si(CH₃)₂), 0.93 (s, 9H, SiC(CH₃)₃), 1.32 (s, 3H, CH₃), 1.55 (s, 3H, CH₃), 2.55 (m, 1H, CH4′), 3.78-3.97 (m, 2H, CH₂OTBDMS), 4.25 (t, 1H, J=7.0 Hz, H3′), 4.48 (t, 1H, J=7.0 Hz, H1′), 4.98-5.02 (m, 1H, H2′), 5.80 (s, 2H, NH₂), 5.90-6.07 (m, 2H, H5′; H6′), 7.87 (s, 1H, H8), 8.37 (s, 1H, H2); ¹³C NMR (CDCl₃) δ −5.8 (Si(CH₃)), −5.6 (Si(CH₃)), 18.1 (SiC(CH₃)₃), 25.4 ((O)₂C(CH₃)), 25.7 (C(CH₃)₃), 27.4 ((O)₂C(CH₃)), 42.9 (CH, C4′), 56.7 (CH, C1′), 63.7 (CH₂OTBDMS), 73.7 (CH, C2′), 76.2 (CH, C3′), 109.2 ((O)₂C(CH₃)), 119.0 (CH, C5), 126.4 (CH, C6′), 130.9 (CH, C5′), 139.9 (CH, C8), 152.9 (CH, C2), 155.5 (C, C6); FAB⁺ 432.2 (M+H)⁺; HRMS calculated for C₂₁H₃₄N₅O₃Si (M+H)⁺ 432.2431, Found 432.2428.

### (±)-(1R,2S,3R,6R)-3-(6-Amino-9H-purin-9-yl)-6-(hydroxymethyl)-4-cyclohexene-1,2-diol (18) (=“ribo”-type cyclohexenyl adenine=rCe-A)

Compound 17 (150 mg, 0.35 mmol) was treated with TFA-H₂O (3:1, 7 mL) at room temperature overnight. The reaction mixture was concentrated and coevaporated with toluene (3×). The residue was chromatographed on silicagel (CH₂Cl₂-MeOH, 9:1, 8:1, 7:1, 7:3) to afford 18 (77 mg, 80.2%) as a yellow-white solid. R_{f} 0.16 (CH₂Cl₂:MeOH 6:1); ¹H NMR (DMSO) (500 MHz) δ 2.41 (m, 1H, H4′), 3.65 (m, 2H, H7′a, H7′b,), 3.94 (m, 1H, H3′), 4.74 (m, 1H, H2′), 4.76 (m, 2H, 7′-OH, 3′-OH), 5.07 (m, 1H, H1′), 5.60 (dt, 1H, j=4.2 Hz, 1.0 Hz, H6′), 5.82 (dt, 1H, J=3.8 Hz, 1.2 Hz, H5′), 7.14 (br-s, 2H, NH₂), 8.02 (s, 1H, H8), 8.12 (s, 1H, H2); ¹³C NMR (DMSO) (500 MHz) δ 45.7 (CH, C4′), 55.3 (CH, C1′), 61.8 (CH, C7′), 67.9 (CH, C3′), 69.4 (CH, C2′), 119.2 (C, C5), 124.5 (CH, C6′), 131.2 (CH, C5′), 140.0 (CH, C8), 149.7 (C, C4), 152.2 (CH, C2), 156.0 (C, C6); FAB⁺ 278.1 (M+H)⁺; HRMS calculated for C₁₂H₁₅N₅O₃ (M+H)⁺ 277.1175, Found 278.1250.

### (±)-9-[(1R,4R,5R,6S)-5,6-Dihydroxy-4-(hydroxymethyl)-2-cyclohexen-1-yl]-1,9-dihydro-6H-purin-6-one (19)

5 mg (0.018 mmol) of racemic 18 was dissolved in 1 mL of hot water. The solution was cooled to room temperature, ADA (10 μL suspension containing 50 units adenosine deaminase) was added in one portion and the reaction mixture was stirred at room temperature overnight. The reaction mixture was concentrated and dissolved in 1 mL of EtOH. 20 μL of this solution was examined by TLC (CH₂Cl₂-MeOH 7:1) and HPLC, which shows that only one of the enantiomers was deaminated. The reacted enantiomer was identified as a hypoxanthine congener.

### Example 7

### Synthesis of the C₂₋alkoxycyclohexenyl Nucleoside Analogs

For the synthesis of the C₂-alkoxy cyclohexenyl nucleoside analogs, the following reaction scheme can be used, as exemplified with adenine.

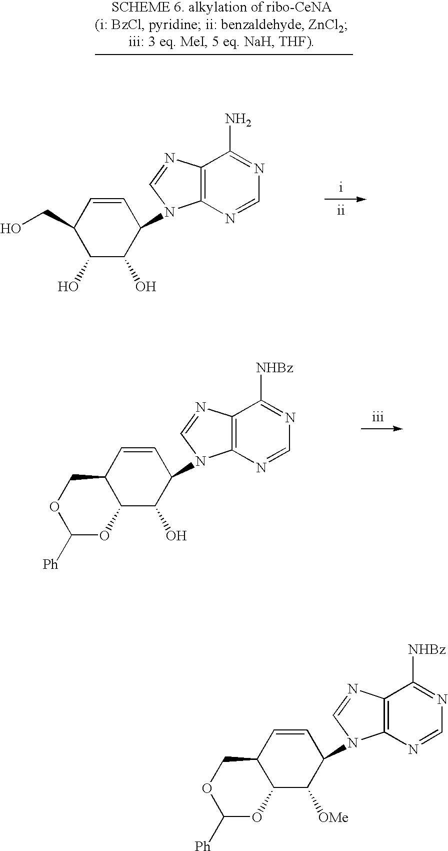

As a final step, deprotection of the benzylidene with 90% TFA will results in the desired compound.

Alkylation with other alkyl groups or other substituents can be performed in an analoguous way.

1. A composition comprising a first oligomer and a second oligomer, wherein:
at least a portion of said first oligomer is capable of hybridizing with at least a portion of said second oligomer,
at least a portion of said first oligomer is complementary to and capable of hybridizing with a selected target nucleic acid, and
both said first and said second oligomer include at least one 6-membered ring containing nucleotide.

2. The composition of claim 1, wherein said first and said second oligomers are a complementary pair of siRNA oligomers.

3. The composition of claim 1, wherein said first oligomer is an antisense oligomer.

4. The composition of claim 1, wherein said second oligomer is a sense oligomer.

5. The composition of claim 1, wherein said first and said second oligomers are an antisense/sense pair of oligomers.

6. The composition of claim 5, wherein the antisense oligomer comprises exactly one 6-membered ring containing nucleotide and the sense oligomer comprises at least one 6-membered ring containing nucleotide.

7. The composition of claim 5, wherein both the antisense and the sense oligomer comprise exactly one 6-membered ring containing nucleotide.

8. The composition of claim 7, wherein the 6-membered ring containing nucleotide is comprised in the middle section of the first oligomer and/or the second oligomer.

9. The composition of claim 1, wherein each of said first and second oligomers has 10 to 40 nucleobases.

10. The composition of claim 1, wherein the first and second oligomer are comprised in one single molecule.

11. An oligomer having at least a first portion and a second portion wherein:
said first portion of said oligomer complementary to and capable of hybridizing with said second portion of said oligomer,
at least a portion of said oligomer complementary to and capable of hybridizing to a selected target nucleic acid, and
each portion of said oligomer comprises at least one 6-membered ring containing nucleotide.

12. An oligomer comprising exactly one 6-membered ring containing nucleotide.

13. The oligomer of claim 12, which is a duplex oligomer.

14. A duplex oligomer, wherein at least one of the strands of said duplex comprises exactly one 6-membered ring containing nucleotide.

15. The duplex oligomer of claim 14, wherein both strands of said duplex comprise exactly one 6-membered ring containing nucleotide.

16. The oligomer of claim 12 wherein said 6-membered ring containing nucleotide is comprised in the middle section of the strand(s).

17. A duplex oligomer comprising in at least one of its strands at least one 6-membered ring containing nucleotide, said 6-membered ring containing nucleotide, said 6-membered ring containing nucleotide contained in the middle section of said strand(s).

18. The duplex oligomer of claim 17, wherein said 6-membered ring containing nucleotide is comprised in the middle section of both strands.

19. The duplex oligomer of claim 18, further comprising additional modified nucleotides in the middle section or any other section of the strands.

20. The duplex oligomer of claim 17, wherein the antisense strand comprises exactly one 6-membered ring containing nucleotide.

21. The duplex oligomer of claim 17, wherein the antisense strand comprise exactly one 6-membered ring containing nucleotide and the sense strand at least one 6-membered ring containing nucleotide.

22. The duplex oligomer of claim of claim 17, wherein both the sense and the antisense strand comprise exactly one 6-membered ring containing nucleotide.

23. The composition of claim 1, wherein said 6-membered ring containing nucleotide is selected from the group consisting of ribo-cyclohexenyl nucleotides, O-substituted ribo-cyclohexenyl nucleotides, altritol nucleotides, O-substituted altritol nucleotides, hexitol ribonucleotides, cyclohexenyl nucleotides, and of any mixture thereof.

24. The composition of claim 23, wherein said 6-membered ring containing nucleotide is selected from the group consisting of ribo-cyclohexenyl nucleotides, O-substituted ribo-cyclohexenyl nucleotides, altritol nucleotides, O-substituted altritol nucleotides and any mixture thereof.

25. A composition comprising a first oligomer and a second oligomer, wherein:
at least a portion of said first oligomer is capable of hybridizing with at least a portion of said second oligomer,
at least a portion of said first oligomer is complementary to and capable of hybridizing with a selected target nucleic acid, and
said first oligomer and/or said second oligomer include at least one 6-membered ring containing nucleotide selected from the group consisting of ribo-cyclohexenyl nucleotides, O-substituted ribo-cyclohexenyl nucleotides, altritol nucleotides, O-substituted altritol nucleotides and any mixture thereof.

26. The composition of claim 25, wherein the antisense oligomer comprises exactly one 6-membered ring containing nucleotide and the sense oligomer comprises at least one 6-membered ring containing nucleotide.

27. The composition of claim 25, wherein both the antisense and the sense oligomer comprise exactly one 6-membered ring containing nucleotide.

28. The composition of claim 23, wherein the at least one 6-membered ring containing nucleotide is comprised in the middle section of the first oligomer and/or the second oligomer.

29. The oligomer of claim 28, further comprising additional modified nucleotides in the middle section or any other section of the strands.

30. The composition of claim 23, wherein each of said first and second oligomers has 10 to 40 nucleobases.

31. The composition of claim 23, wherein the first and second oligomer are comprised in one single molecule.

32. The composition of claim 1, wherein said 6-membered ring containing nucleotide is a hexitol nucleotide according to formula I, 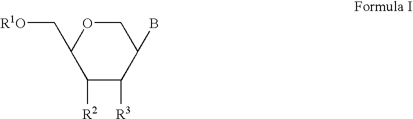
wherein
B is a substituted or unsubstituted heterocyclic ring;
R¹ is independently selected from H, an internucleotide linkage to an adjacent nucleotide or a terminal group;
R² is independently selected from the group consisting of phosphate, from any modification known for nucleotides to replace the phosphate group, from an internucleotide linkage to an adjacent nucleotide and a terminal group;
R³ is independently selected from the group consisting of H, alkyl group, alkenyl group, alkynyl group, azido group, F, Cl, I, substituted or unsubstituted amino, OR⁴, SR⁴, aroyl, alkanoyl and any substituent known for modified nucleotides;
R⁴ is selected from the group consisting of hydrogen; alkyl group; alkenyl group; alkynyl group; wherein said alkyl group, alkenyl group and alkynyl group can contain one or more heteroatoms in or at the end of the hydrocarbon chain, said heteroatom selected from O, S and N and salts, esters and isomers thereof.

33. The composition of claim 1, wherein said 6-membered ring containing nucleotide is a cyclohexenyl nucleotide according to formula II 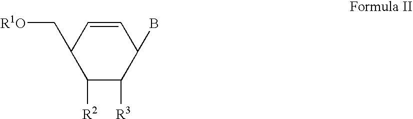
wherein
B is a substituted or unsubstituted heterocyclic ring;
R¹ is independently selected from the group consisting of H, an internucleotide linkage to an adjacent nucleotide or a terminal group;
R² is independently selected from the group consisting of phosphate, any modification known for nucleotides to replace the phosphate group, or from an internucleotide linkage to an adjacent nucleotide or a terminal group;
R³ is independently selected from the group consisting of H; OH; O-alkyl; O-alkenyl, or O-alkynyl or O-acyl, wherein said alkyl, alkenyl and alkynyl can contain one or more heteroatoms in or at the end of the hydrocarbon chain, said heteroatom selected from O, S and N and salts, esters and isomers thereof.

34. The composition of claim 1, wherein said 6-membered ring containing nucleotide is a ribo-cyclohexenyl nucleotide according to formula III 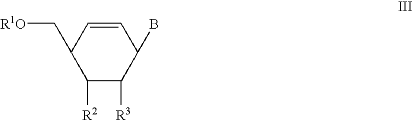
wherein
B is a substituted or unsubstituted heterocyclic ring;
R¹ is independently selected from the group consisting of H; alkyl; alkenyl; alkynyl; acyl; phosphate moieties and a protecting group;
R² is independently selected from the group consisting of OH, phosphate, any modification known for nucleotides to replace the phosphate group, or from an internucleotide linkage to an adjacent nucleotide or a terminal group;
R³ is independently selected from the group consisting of H, OH; O-alkyl; O-alkenyl; O-alkynyl; O-acyl, wherein said alkyl, alkenyl group and alkynyl group can contain one or more heteroatoms in or at the end of the hydrocarbon chain, said heteroatom selected from O, S and N and salts, esters and isomers thereof.

35. A 6-membered ring containing nucleoside or nucleotide which is a C₂-substituted cyclonexenly nucleoside or nucleotide.

36. The nucleoside or nucleotide of claim 35 selected from the group consisting of a ribo-cyclohexenyl nucleoside, a ribo-cyclohexenyl nucleotide, a C₂—O-substituted ribo-cyclohexenyl nucleoside and a C₂—O-substituted ribo-cyclohexenyl nucleotide.

37. The nucleotide or nucleoside according to claim 35, wherein the nucleoside or nucleotide is one according to formula III 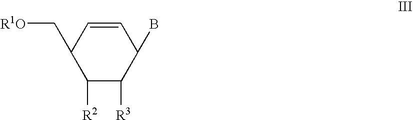
wherein
B is a substituted or unsubstituted heterocyclic ring;
R¹ is independently selected from the group consisting of H; alkyl; alkenyl; alkynyl; acyl; phosphate moieties and a protecting group;
R² is independently selected from the group consisting of OH; O-alkyl; O-alkenyl; O-alkynyl; O-acyl; a O-protecting group phosphate; any modification known for nucleotides to replace the phosphate group, or from an internucleotide linkage to an adjacent nucleotide or a terminal group, wherein said alkyl, alkenyl and alkynyl can contain one or more heteroatoms in or at the end of the hydrocarbon chain, said heteroatom selected from O, S and N;
R³ is independently selected from the group consisting of OH; O-alkyl; O-alkenyl; O-alkynyl; O-acyl and an O-protecting group, wherein said alkyl, alkenyl and alkynyl can contain one or more heteroatoms in or at the end of the hydrocarbon chain, said heteroatom selected from O, S and N (and isomers salts or esters thereof).

38. (canceled)

39. A nucleotide sequence comprising at least one nucleoside or nucleotide according to claim 35.

40. The nucleotide sequence of claim 39, which is an oligomer.

41. The nucleotide sequence of claim 10, which is a siRNA molecule, a miRNA molecule or a shRNA molecule.

42. A pharmaceutical composition comprising an element selected from the group, consisting of the an oligomer of claim 11, the composition of claim 1 or the nucleotide of claim 36, and a pharmaceutically acceptable carrier.

43. A method of modulating the expression of a target nucleic acid in a cell, comprising the step of contacting said cell with the composition according to claim 42.

44. The method of claim 43, wherein said method inhibits or decreases expression of a target nucleic acid compared to a control.

45. The method of claim 44, wherein the expression of P-glycoprotein efflux pumps is inhibited or decreased.

46. The method of claim 44, wherein the expression of the MDR1 gene is down-regulated.

47. A method of treating or preventing a disease or disorder associated with a target nucleic acid, comprising the step of administering to an animal having or predisposed to said disease or disorder a therapeutically effective amount of the composition claim 42.

48. (canceled)

49. The method of claim 47, wherein said disease or disorder is cancer.

50. A duplex oligomer according to claim 17, which is a siRNA, a miRNA or a shRNA duplex.

51. An intermediates used during the course of manufacturing one or more of the C₂-substituted cyclohexenyl nucleosides of the formula III, represented by one of the formulae IV, V (also Va, Vb and Vc), VI, VII and VIIIa: 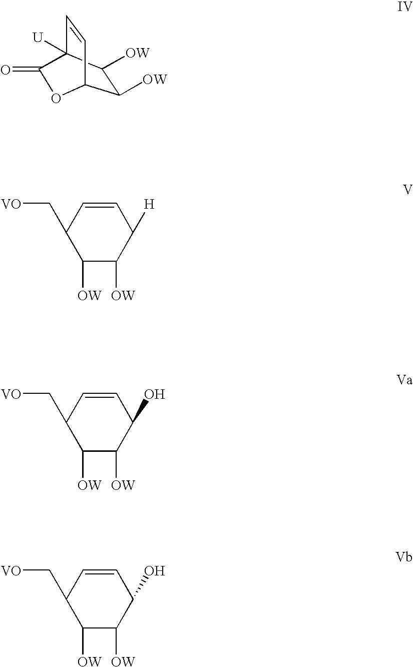 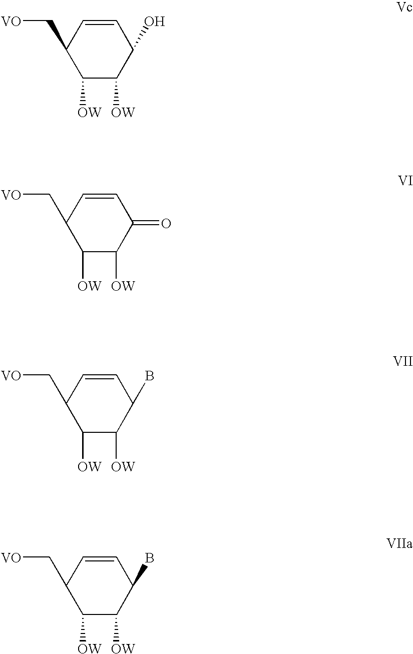
wherein
U is selected from the group consisting of hydrogen and halogen;
W represents a protecting group, the group consisting of an acetal and ketal protecting the neighbouring diol;
V is selected from the group consisting of hydrogen and a protecting group;
B is selected from a substituted or unsubstituted heterocyclic ring.

52. The nucleotide sequence of claim 40, which is a duplex oligomer.
